(19) United States
(12) Patent Application Publication
(10) Pub. No.: US 2026/0347819 A1
(43) Pub. Date: Nov. 5, 2026

(54) FOOD SUPPLEMENT COMPOSITION TO AID IN DIGESTION OF FRUCTAN, MANNITOL, AND SORBITOL

(71) Applicant: KIWI HEALTH, INC, Somerville, MA (US)

(72) Inventor: Anjie LIU, Somerville, MA (US)

(73) Assignee: KIWI HEALTH, INC, Somerville, MA (US)

(21) Appl. No.: 19/735,793

(22) Filed: Jul. 9, 2026

Related U.S. Application Data
(62) Division of application No. 18/005,280, filed on Jul. 23, 2021, now abandoned.
(60) Provisional application No. 63/055,874, filed on Jul. 23, 2020.
(60) Provisional application No. 63/105,958, filed on Oct. 27, 2020.
(60) Provisional application No. 63/106,437, filed on Oct. 28, 2020.
(60) Provisional application No. 63/165,201, filed on Mar. 24, 2021.
(60) Provisional application No. 63/177,451, filed on Apr. 21, 2021.

(51) Int. Cl. C12N 9/24; A23L 33/17; A61P 1/14; C12N 9/04; C12N 9/40; C12N 9/92

(52) U.S. Cl. CPC C12N 9/2482; A23L 33/17; A61P 1/14; C12N 9/0006; C12N 9/2465; C12N 9/92; C12Y 101/01014; C12Y 101/01067; C12Y 302/01007

(57) ABSTRACT

The technology described herein is directed to combinations and combinations for reducing the symptoms of digestive conditions, e.g., caused by ingestion of FODMAPs.

### CROSS-REFERENCE TO RELATED APPLICATIONS

This application is a divisional under 35 U.S.C. § 121 of U.S. Ser. No. 18/005,280 filed Jan. 12, 2023, which is a 35 U.S.C. § 371 National Phase Entry Application of International Application No. PCT/US2021/042879 filed Jul. 23, 2021, which designates the U.S. and claims benefit under 35 U.S.C. § 119(e) of U.S. Provisional Application No. 63/055,874 filed Jul. 23, 2020; 63/105,958 filed Oct. 27, 2020; 63/106,437 filed Oct. 28, 2020; 63/165,201 filed Mar. 24, 2021; and 63/177,451 filed Apr. 21, 2021, the contents of which are incorporated herein by reference in their entireties.

### SEQUENCE LISTING

The instant application contains a Sequence Listing which has been submitted electronically in XML format and is hereby incorporated by reference in its entirety. Said XML copy, created on Jun. 30, 2026, is named 088866-098230USD1_SL.xml and is 1,275,397 bytes in size.

### TECHNICAL FIELD

The technology described herein relates to food supplements to aid digestion.

### BACKGROUND

Many individuals, both with or without a diagnosed digestive condition, find certain foodstuffs difficult to consume. For example, certain foods can cause gas, bloating, abdominal pain, nausea, diarrhea, and/or constipation. One reason for such reaction is the presence in food of the compounds referred to as FODMAPs (fermentable oligo-, di-, monosaccharides, and polyols), a wide group of carbohydrates that includes fructans, sorbitol, and mannitol.

Fructan is a polysaccharide polymer composed of a chain of fructose molecules and typically a terminal glucose molecule. Inulin is a common type of fructan and is found in a number of common foods such as garlic, onion, banana, dates, watermelon, wheat, and barley.

The human body does not produce endogenous enzymes that target fructans as substrates. Instead, fructans, including inulin, are typically transported uncleaved and intact through the gastrointestinal tract. Along the way, they accumulate water via osmotic effect (essentially, they act in a sponge-like manner) in the intestines and become fermented by microorganisms in the lower intestines, which may produce painful or uncomfortable symptoms including but not limited to gas, bloating, distension, nausea, and impaired bowel motility, resulting in diarrhea or constipation in sensitive individuals or individuals intolerant to fructans who may be suffering from IBS, IBD, SIBO, SIFO, or other gastrointestinal conditions.

Sorbitol and mannitol are sugar alcohols, a class of polyols. Sorbitol is found in high levels in foods such as apples, pears, peaches, nectarines, apricots, and avocados. Cauliflower, celery, and several types of mushrooms contain high amounts of mannitol.

The human body does not readily absorb sorbitol and mannitol, nor does it produce enzymes that convert them to molecules more readily absorbed by the digestive tract. Similar to fructans, sorbitol and mannitol can have significant osmotic effects in the gastrointestinal tract. In the scientific literature, it has been noted that mannitol has been found to rapidly increase water load in the small intestine by up to ten times more than that effectuated by an equal amount of glucose. Sorbitol and mannitol are also commonly fermented in the lower intestines. The osmotic properties and fermentability of these molecules may produce painful or unpleasant symptoms similar to those produced by the osmotic properties and fermentability of fructans described above.

Hitherto, the established and widely accepted clinical protocol for individuals suffering from the associated afflictions is to advise such individuals to simply avoid foods and beverages that contain a significant quantity of the symptom-triggering substances. In particular, individuals suffering from illnesses including but not limited to IBS, IBD, SIBO, and SIFO are often recommended to follow a diet low in FODMAPs. Typically, it is recommended that the afflicted individuals adhere strictly to a low-FODMAP diet for several weeks; then, a period of reintroduction of various food groups (such as fructans, sorbitol, or mannitol) within the FODMAPs designation follows. During this period, certain individuals may find that they are particularly sensitive to particular classes of FODMAPs, but not to others. As a consequence, the afflicted individuals are advised to avoid foods and beverages containing sufficiently high levels of the specific FODMAP classes to which they are sensitive.

The shortcomings of such an approach are numerous. Some of the most obvious disadvantages in the approach are the deprivation of gastronomical enjoyment, restriction on the variety of nutritional sources, and dietary inconvenience. An afflicted individual, under the existing therapeutic protocol, would be doomed to a cumbersome and deprived lifestyle of constant inspection of ingredient labels on food products and inquiry into the composition of restaurant offerings or catered meals (which still leaves the individual vulnerable to mistakes on the food supplier's part). An individual sensitive to fructans, for instance, would, at each meal, carry the responsibility of quantifying the amount of fructans in his or her intake and ensuring that the quantity is below recommended cutoff values. Often, safe ranges for certain foods are extremely narrow. For instance, the Monash University FODMAP smartphone application, with the largest FODMAP food database available to date, indicates a 0.35 oz serving of canned corned kernels as sufficiently low in fructans, thus safe (indicated by a green circle), but a 0.53 oz (approximately one tablespoon) serving as containing a moderate amount of fructans, and thus risky (indicated by a yellow circle) for consumption. Furthermore, quantities of FODMAPs such as fructan, sorbitol, and mannitol add up, and quantities of foods considered “safe” over multiple servings of various food types may accumulate to “risky” levels. The database does not to date offer a means to quantify FODMAP content, and even if it were to, it would still be incredibly tedious and frustrating to keep track of FODMAP content at every meal.

The psychological difficulty in practicing the restraint required for such a lifestyle is considerably high. For instance, one must resist culinary temptations offered in social settings and refrain from taking one more mouthful of a cherished food. Moreover, there currently exist significant knowledge gaps in the understanding of FODMAPs. The Monash University FODMAP application, though broad in its range of foods covered, is certainly far from comprehensive. Exotic fruits or vegetables encountered during travels may not be documented by the application. Moreover, preparations of certain food products may vary greatly by producer and/or country of origin. For instance, German Bratwurst sausage is listed as high in fructans (indicated by a red circle) at a serving of 7.05 oz, but tolerable at 3.53 oz. However, there are multiple ways in which German Bratwurst sausage may be prepared, for instance, from little or no garlic in the recipe to preparations with much garlic (0.11 oz of garlic is considered a serving high in fructans). The application database alone, in this scenario, will leave an afflicted individual sensitive to fructans ill-equipped to determine a reasonably safe quantity of German Bratwurst sausage to consume.

The use of an enzyme exhibiting fructan hydrolase activity and the production of an enzyme derived from a strain of Lactobacillus has been described (US20190174773), specifically the use of said enzyme in the degradation of fructan in grain and vegetable raw material. The drawbacks of such a method are such that the method can only reasonably be applied to powdered, liquid, puréed, grounded, or otherwise homogenized foodstuff. One cannot apply the enzyme to a whole ripe banana, for instance, or to a bulk of leek. Accordingly, the culinary application of this method is very much limited. Not only is the range of foods which can possibly be treated via this method limited to specific textures and preparations of ingredients, but even so, it is unreasonable to expect that the variety of food products prepared via this method will come anywhere close to the variety of ordinarily prepared (that is, with normal levels of fructan content) food products. Hence, an afflicted individual will still be severely restricted in choosing food products available to them at safe levels of fructan content, whether in the supermarket, at restaurants, social gatherings, and other dining events not specifically catered to individuals with fructan sensitivities.

Therefore, what is lacking and desired in the art is a method for individuals with fructan intolerance or sensitivity, or, more generally, issues with the presence of fructans in their digestive tracts, to break down fructan molecules and absorb the component molecules in whatever foods they may consume, regardless of texture, preparation, or composition, before symptoms arise, in any setting they may choose to enjoy their dining or snacking experience. Similarly, a method is needed for individuals sensitive or intolerant to sorbitol and/or mannitol to be able to enjoy foods and beverages containing the molecules without the unpleasant and/or painful consequences. The present disclosure is intended to provide such methods as described herein.

### SUMMARY

As noted above, humans do not readily absorb fructans, sorbitol, and mannitol, nor do they endogenously secrete enzymes that break down or convert these molecules to more readily digestible molecules in the digestive tract. Fructans, sorbitol, and mannitol typically pass through the gastrointestinal tract undigested to be fermented by colonic microbiota, which may cause painful bloating and gas, and may disrupt regularity of bowel movements in individuals sensitive to these substances and/or afflicted by IBS, IBD, SIBO, SIFO or other gastrointestinal or digestive maladies. Additionally, fructans, sorbitol, and mannitol may generate osmotic activity in the small and large intestines, inducing bloating and distention discomfort, as well as negatively affecting bowel motility.

The present invention relates to the field of gastrointestinal relief. Specifically, the present invention relates in some embodiments to a dietary supplement composition containing at least one of the following enzymes: inulinase, sorbitol dehydrogenase, and mannitol dehydrogenase. Inulinase is an enzyme that cleaves inulin, a common form of fructan, ultimately into glucose and fructose. Sorbitol dehydrogenase is an enzyme that is able to convert sorbitol to fructose. Similarly, mannitol dehydrogenase is able to convert mannitol to fructose and/or mannose.

Embodiments of the invention can help individuals afflicted with intolerance of or sensitivity to fructans, sorbitol, mannitol, or a combination of these molecules by converting those molecules in the gastrointestinal tract into more easily absorbed molecules. Fructans, sorbitol, and mannitol are known to cause gastrointestinal troubles such as, but not limited to, bloating, gas, nausea, abdominal pain, diarrhea, and constipation in individuals suffering from gastrointestinal or digestive ailments including but not limited to Irritable Bowel Syndrome (IBS), Inflammatory Bowel Disease (IBD), Small Intestinal Bacterial Overgrowth (SIBO), and Small Intestinal Fungal Overgrowth (SIFO).

It is further noted that the compositions and methods described herein provide multiple advantages over prior approaches to FODMAPs. In particular, the prior art describes attempts to treat foodstuffs during manufacturing in order to reduce or remove FODMAPs. Such treatments often alter the taste of foods, causing them to taste sweeter. In contract, the compositions and methods described herein can provide relief from symptoms caused by FODMAPs without altering the taste of the foodstuffs consumed, as the FODMAP content is not altered prior to consumption. In addition, the compositions and methods described herein can be used when dining in restaurants, cafeterias, or social settings, where the user will not have access to specialty ingredients that have been pretreated to reduce or remove FODMAPs. The compositions and methods described herein therefore offer increased relief from symptoms as they can be used more often and without the social isolation and inconvenience caused by a need to avoid restaurants, cafeterias, or meals in social settings outside the user's home.

It is specifically noted that experts in the field have repeatedly urged that oral consumption of various enzymes that target one or more FODMAPs is not expected to address FODMAP sensitivities, particularly in relation to fructans and polyols. These experts have urged that the gastrointestinal tract conditions will not permit sufficient hydrolysis to relieve symptoms, or that the relevant FODMAPS are such relatively simple molecules that enzymes either cannot break them down or induce a change that will impact digestion. FODMAP sensitivities have long been known to medical science, but leading medical experts continue to insist on low FODMAP diets as the suitable treatment or to focus on food processing approaches as new techniques. Oral administration of therapeutic compositions to treat FODMAP sensitivities has been repeatedly discouraged and disparaged. As demonstrated in the Examples herein, the inventor has surprisingly found that orally-consumed compositions containing fructan-degrading enzymes actually exhibit striking therapeutic efficacy, reducing and in some cases entirely eliminating symptoms of FODMAP sensitivity. In some embodiments of any of the aspects, the present invention relates to a composition and method for degrading fructans and converting sorbitol and mannitol present in common food sources into molecules absorbable by the gastrointestinal tract before the fructan, sorbitol, and mannitol molecules are able to substantially inflict gastrointestinal distress including but not limited to the symptoms listed above.

In one aspect of any of the embodiments, provided herein is a composition or combination comprising, consisting of, or consisting essentially of at least one of:
a. At least one fructan-degrading enzyme;
b. At least one galactosidase;
c. At least one sorbitol-degrading enzyme;
d. At least one mannitol-degrading enzyme;
e. At least one glucose isomerase; and
f. At least one mannose isomerase.

In one aspect of any of the embodiments, provided herein is composition or combination comprising, consisting of, or consisting essentially of at least one of:
a. At least one fructan-degrading enzyme;
b. At least one galactosidase;
c. At least one sorbitol-degrading enzyme;
d. At least one mannitol-degrading enzyme; and
e. At least one glucose isomerase.

In some embodiments of any of the aspects, the composition or combination comprises least one, two, three, or four of:
a. At least one fructan-degrading enzyme;
b. At least one galactosidase;
c. At least one sorbitol-degrading enzyme; and
d. At least one mannitol-degrading enzyme.

In some embodiments of any of the aspects, the composition or combination comprises at least one fructan-degrading enzyme; and at least one galactosidase. In some embodiments of any of the aspects, the composition or combination comprises at least one fructan-degrading enzyme; at least one sorbitol-degrading enzyme; at least one mannitol-degrading enzyme; and at least one glucose isomerase.

In one aspect of any of the embodiments, provided herein is a composition or combination comprising, consisting of, or consisting essentially of:
a. At least one fructan-degrading enzyme;
b. At least one galactosidase;
c. At least one sorbitol-degrading enzyme;
d. At least one mannitol-degrading enzyme;
e. At least one glucose isomerase; and
f. At least one mannose isomerase.

In some embodiments of any of the aspects, the at least one fructan-degrading enzyme comprises an inulinase, e.g., a yeast inulinase. In some embodiments of any of the aspects, the inulinase is an exo-inulinase. In some embodiments of any of the aspects, the exo-inulinase is an exo-inulinase of, or is obtained from an Aspergillus or Kluyveromyces spp. In some embodiments of any of the aspects, the exo-inulinase is an exo-inulinase of, or is obtained from Aspergillus oryzae or Kluyveromyces marxianus. In some embodiments of any of the aspects, the exo-inulinase is an exo-inulinase of, or is obtained from Kluyveromyces marxianus CBS6014. In some embodiments of any of the aspects, the exo-inulinase has a sequence with at least 80% sequence identity to one of SEQ ID NOs: 3-7, 29-31, and 42-45. In some embodiments of any of the aspects, the inulinase is an endo-inulinase. In some embodiments of any of the aspects, the endo-inulinase is an endo-inulinase of, or is obtained from an Aspergillus spp. In some embodiments of any of the aspects, the endo-inulinase is an endo-inulinase of, or is obtained from Aspergillus niger. In some embodiments of any of the aspects, the endo-inulinase has a sequence with at least 80% sequence identity to one of SEQ ID NOs: 1-2. In some embodiments of any of the aspects, the composition or combination comprises both an exo-inulinase and an endo-inulinase.

In some embodiments of any of the aspects, the at least one fructan-degrading enzyme comprises an amyloglucosidase. In some embodiments of any of the aspects, the amyloglucosidase comprises a sequence with at least 80% sequence identity to one of SEQ ID NOs: 8-10.

In some embodiments of any of the aspects, the at least one galactosidase comprises an alpha-galactosidase. In some embodiments of any of the aspects, the alpha-galactosidase is an alpha-galactosidase of, or is obtained from an Aspergillus spp. In some embodiments of any of the aspects, the alpha-galactosidase is an alpha-galactosidase of, or is obtained from Aspergillus niger. In some embodiments of any of the aspects, the alpha-galactosidase comprises a sequence with at least 80% sequence identity to one of SEQ ID NOs: 11-14.

In some embodiments of any of the aspects, the at least one galactosidase comprises a beta-galactosidase. In some embodiments of any of the aspects, the beta-galactosidase is a beta-galactosidase of, or is obtained from an Aspergillus spp. In some embodiments of any of the aspects, the beta-galactosidase is a beta-galactosidase of, or is obtained from Aspergillus niger. In some embodiments of any of the aspects, the beta-galactosidase comprises a sequence with at least 80% sequence identity to SEQ ID NO: 15.

In some embodiments of any of the aspects, the at least one galactosidase comprises an alpha-galactosidase and a beta-galactosidase.

In some embodiments of any of the aspects, the at least one sorbitol-degrading enzyme comprises a sorbitol dehydrogenase. In some embodiments of any of the aspects, the sorbitol dehydrogenase is a mammalian sorbitol dehydrogenase. In some embodiments of any of the aspects, the sorbitol dehydrogenase is a sorbitol dehydrogenase of, or is obtained from a Ovis or Rattus species. In some embodiments of any of the aspects, the sorbitol dehydrogenase has a sequence with at least 80% sequence identity to one of SEQ ID NOs: 16-18. In some embodiments of any of the aspects, the at least one sorbitol-degrading enzyme comprises a sorbitol oxidase. In some embodiments of any of the aspects, the sorbitol oxidase is a yeast sorbitol oxidase. In some embodiments of any of the aspects, the sorbitol oxidase has a sequence with at least 80% sequence identity to SEQ ID NO: 19.

In some embodiments of any of the aspects, the at least one mannitol-degrading enzyme comprises a mannitol dehydrogenase. In some embodiments of any of the aspects, the mannitol dehydrogenase is a bacterial mannitol dehydrogenase. In some embodiments of any of the aspects, the mannitol dehydrogenase is a mannitol dehydrogenase of, or is obtained from a Pseudomonas, Escherichia, Leuconostoc, or Gluconobacter spp. In some embodiments of any of the aspects, the mannitol dehydrogenase is a mannitol dehydrogenase of, or is obtained from P. fluorescens, E. coli, L. mesenteroides, or G. oxydans. In some embodiments of any of the aspects, the mannitol dehydrogenase has a sequence with at least 80% sequence identity to one of SEQ ID NOs: 20-26. In some embodiments of any of the aspects, the at least one mannitol-degrading enzyme comprises a mannitol oxidase.

In some embodiments of any of the aspects, the glucose isomerase is a yeast glucose isomerase. In some embodiments of any of the aspects, the glucose isomerase is a glucose isomerase of, or is obtained from a Streptomyces spp. In some embodiments of any of the aspects, the glucose isomerase is a glucose isomerase of, or is obtained from S. murinus. In some embodiments of any of the aspects, the glucose isomerase has a sequence with at least 80% sequence identity to SEQ ID NO: 27.

In some embodiments of any of the aspects, the mannose isomerase is a bacterial mannose isomerase. In some embodiments of any of the aspects, the mannose isomerase is a mannose isomerase of, or is obtained from a Thermobifida spp. In some embodiments of any of the aspects, the mannose isomerase is a mannose isomerase of, or is obtained from T. fusca. In some embodiments of any of the aspects, the mannose isomerase has a sequence with at least 80% sequence identity to SEQ ID NO: 28.

In some embodiments of any of the aspects, the composition or combination further comprises one or more of: a cofactor; a metal ion; an antacid; an H2 antagonist; a proton pump inhibitor; a pepstatin protease inhibitor; or a protease inhibitor. In some embodiments of any of the aspects, the composition or combination is formulated as a food supplement. In some embodiments of any of the aspects, the composition or combination further comprises a carrier, e.g., a pharmaceutically acceptable carrier. In some embodiments of any of the aspects, the composition or combination is provided in a shelf-stable formulation. In some embodiments of any of the aspects, the composition or combination is formulated as a powder, a solution, a pill, a capsule, a concentrate, a tablet, a soft-gel, a thin-film, a liquid, and/or a syrup. In some embodiments of any of the aspects, the composition or combination is formulated with an enteric coating. In some embodiments of any of the aspects, the composition or combination is provided as a single composition. In some embodiments of any of the aspects, the composition or combination is provided as a combination of multiple compositions that can be mixed prior to administration or consumption, and/or administered or consumed separately.

In some embodiments of any of the aspects, the composition or combination does not comprise a live microbe. In some embodiments of any of the aspects, the composition or combination does not comprise a live yeast. In some embodiments of any of the aspects, the composition or combination does not comprise flour. In some embodiments of any of the aspects, the composition or combination does not comprise an invertase, a beta-fructofuranosidase, alpha-galactosidease, beta-glucanase, and/or pectinase. In some embodiments of any of the aspects, the composition or combination does further comprise 5-D-fructose dehydrogenase.

In one aspect of any of the embodiments, provided herein is a nucleic acid comprising a sequence encoding one or more of:
a. At least one fructan-degrading enzyme;
b. At least one galactosidase;
c. At least one sorbitol-degrading enzyme;
d. At least one mannitol-degrading enzyme;
e. At least one glucose isomerase; and
f. At least one mannose isomerase; and
at least one of:
g. an operably linked heterologous promoter; and
h. at least one codon optimized for expression in bacteria or yeast.

In one aspect of any of the embodiments, provided herein is a cell comprising the foregoing nucleic acid.

In one aspect of any of the embodiments, provided herein is a method of improving digestion, the method comprising consuming, or administering to a subject, the composition or combination described herein. In one aspect of any of the embodiments, provided herein is a method of treating a digestive condition, the method comprising administering to a subject, the composition or combination described herein. In one aspect of any of the embodiments, provided herein is a composition or combination as described herein for use in a method of treating a digestive condition in a subject.

In some embodiments of any of the aspects, administering the composition to a subject comprises directing the subject to consume the composition. In some embodiments of any of the aspects, consumption or administration occurs within 30 minutes of consumption of a meal, or consumption of other food or drink, e.g., within 30 minutes prior, within 20 minutes prior, within 10 minutes prior, or at at least partially during consumption. In some embodiments of any of the aspects, the digestive condition is Irritable Bowel Syndrome (IBS); Inflammatory Bowel Disease (IBD); Non-celiac gluten sensitivity; small intestinal bacterial overgrowth (SIBO); small intestinal fungal overgrowth (SIFO); and/or allium intolerance. In some embodiments of any of the aspects, the subject is sensitive to one or more FODMAPs. In some embodiments of any of the aspects, bloating, gas, abdominal pain, abdominal discomfort, nausea, diarrhea, and/or constipation is decreased.

### BRIEF DESCRIPTION OF THE DRAWINGS

FIG. 1 depicts a graph of weighted flatulence scores following fructan consumption with or without exo-inulinase.

FIG. 2 depicts graphs of diarrhea episodes and weighted flatulence scores following fructan consumption with or without exo-inulinase.

### DETAILED DESCRIPTION

As described above, the compositions and methods provided herein provide improved digestion of one or more FODMAPs. In one aspect of any of the embodiments, described herein is a composition or combination comprising, consisting of, or consisting essentially of at least one of: a) at least one fructan-degrading enzyme; b) at least one galactosidase, c) at least one sorbitol-degrading enzyme; d) at least one mannitol-degrading enzyme; e) at least one glucose isomerase; f) at least one mannose isomerase, and g) at least one lactase. Any one of the foregoing element options (e.g., a-g), and combinations thereof (e.g., 2, 3, 4, 5, 6, or 7) of the foregoing are specifically contemplated herein. In addition, 2 or more species of any of the individual element options can be used or present in the same composition or combination. For example, 2 or more different fructan-degrading enzymes can be used or present in the same composition or combination.

In one aspect of any of the embodiments, described herein is a composition or combination comprising, consisting of, or consisting essentially of at least one of: at least one fructan-degrading enzyme; at least one galactosidase; at least one sorbitol-degrading enzyme; at least one mannitol-degrading enzyme; and at least one glucose isomerase. In one aspect of any of the embodiments, described herein is a composition or combination comprising, consisting of, or consisting essentially of at least one of: at least one fructan-degrading enzyme; at least one galactosidase; at least one sorbitol-degrading enzyme; and at least one mannitol-degrading enzyme. In one aspect of any of the embodiments, described herein is a composition or combination comprising, consisting of, or consisting essentially of at least two of: at least one fructan-degrading enzyme; at least one galactosidase; at least one sorbitol-degrading enzyme; and at least one mannitol-degrading enzyme. In one aspect of any of the embodiments, described herein is a composition or combination comprising, consisting of, or consisting essentially of at least three of: at least one fructan-degrading enzyme; at least one galactosidase; at least one sorbitol-degrading enzyme; and at least one mannitol-degrading enzyme. In one aspect of any of the embodiments, described herein is a composition or combination comprising, consisting of, or consisting essentially of at least one fructan-degrading enzyme; at least one galactosidase; at least one sorbitol-degrading enzyme; and at least one mannitol-degrading enzyme. In one aspect of any of the embodiments, described herein is a composition or combination comprising, consisting of, or consisting essentially of at least one fructan-degrading enzyme; at least one galactosidase; at least one sorbitol-degrading enzyme; at least one mannitol-degrading enzyme; and at least one glucose isomerase. In one aspect of any of the embodiments, described herein is a composition or combination comprising, consisting of, or consisting essentially of at least one fructan-degrading enzyme; at least one galactosidase; at least one sorbitol-degrading enzyme; at least one mannitol-degrading enzyme; at least one glucose isomerase; and at least one mannose isomerase. In some embodiments of any of the aspects, the composition or combination further comprises at least one lactase.

In one aspect of any of the embodiments, described herein is a composition or combination comprising, consisting of, or consisting essentially of at least one fructan-degrading enzyme and at least one galactosidase. In one aspect of any of the embodiments, described herein is a composition or combination comprising, consisting of, or consisting essentially of at least one fructan-degrading enzyme, at least one galactosidase, and at least one lactase.

In one aspect of any of the embodiments, described herein is a composition or combination comprising, consisting of, or consisting essentially of at least one fructan-degrading enzyme and at least one galactosidase and not comprising a pectinase. In one aspect of any of the embodiments, described herein is a composition or combination comprising, consisting of, or consisting essentially of at least one fructan-degrading enzyme and at least one lactase and not comprising a pectinase. In one aspect of any of the embodiments, described herein is a composition or combination comprising, consisting of, or consisting essentially of at least one fructan-degrading enzyme, at least one galactosidase, and at least one lactase; and not comprising a pectinase. In some embodiments of any of the aspects, the composition or combination does not comprise a glucose isomerase.

In one aspect of any of the embodiments, described herein is a composition or combination comprising, consisting of, or consisting essentially of at least one fructan-degrading enzyme and not comprising a galactosidase. In one aspect of any of the embodiments, described herein is a composition or combination comprising, consisting of, or consisting essentially of at least one fructan-degrading enzyme and not comprising a lactase. In one aspect of any of the embodiments, described herein is a composition or combination comprising, consisting of, or consisting essentially of at least one fructan-degrading enzyme and not comprising a galactosidease or a lactase. In one aspect of any of the embodiments, described herein is a composition or combination comprising, consisting of, or consisting essentially of at least one fructan-degrading enzyme and not comprising a pectinase. In some embodiments of any of the aspects, the composition or combination does not comprise a glucose isomerase.

As used herein, “fructan-degrading enzyme” refers to any enzyme that catabolizes fructan or a catabolic product of fructan. Fructans are a group of polymers comprising fructose units. Fructans include inulins, and levans. The linkage position of the fructose residues determines the type of the fructan. Individual fructans can comprise additional sugars, including by way of non-limiting example, glucose and sucrose.

In some embodiments of any of the aspects, the fructan is inulin. Inulins are polymers comprising fructose units linked by β (2,1) glycosidic bonds and can further comprise a chain-terminating glucose. Inulins can be linear or branched.

Fructan catabolism can comprise:
1) endohydrolysis of (2→1)-beta-D-fructosidic linkages in inulin by inulinase (e.g., endo-inulinase, E.C. 3.2.1.7) or endo-levanases;
2) cleavage of terminal glucose-fructose or fructose-fructose O—C bonds in a fructan polymer by exo-inulinase (also known as beta-fructosidase, fructan beta-fructosidase (e.g., E.C. 3.2.1.80), beta-fructofuranosidase, and invertase (e.g., E.C. 3.2.1.26), see, e.g., SUC2 of S. cerevisiae, NCBI ID: 854644) or exo-levanases to release one fructose or glucose molecule from the fructan; and/or
2) hydrolysis of a terminal glucose from fructan by an amyloglucosidase (AMG) that hydrolyzes an alpha-glycosidic linkage (see, e.g., CAS 9032 August 8, available commercially from Millipore Sigma (St. Louis, MO) under Cat. No. A7420).
   Fructan-degrading enzymes are known in the art as described below herein.

In some embodiments of any of the aspects, the at least one fructan-degrading enzyme comprises, consists of, or consists essentially of one or more inulinases. Inulinases are known in the art and can be, e.g., a yeast inulinase. Sequences of inulinases, e.g., yeast inulinases, can be readily obtained from publicly available databases, e.g., the NCBI and/or UNIPROT databases or obtained commercially. In some embodiments of any of the aspects, the at least one fructan-degrading enzyme comprises, consists of, or consists essentially of one or more levanases. Levanases are known in the art and can be, e.g., a bacterial levanase. Sequences of levanases can be readily obtained from publicly available databases, e.g., the NCBI and/or UNIPROT databases or obtained commercially.

In some embodiments of any of the aspects, the at least one fructan-degrading enzyme comprises, consists of, or consists essentially of one or more endo-inulinases. In some embodiments of any of the aspects, the at least one fructan-degrading enzyme comprises, consists of, or consists essentially of one or more exo-inulinases. In some embodiments of any of the aspects, the at least one fructan-degrading enzyme comprises, consists of, or consists essentially of one or more amyloglucosidases.

As used herein “endo-inulinase” refers to an enzyme that performs endohydrolysis of the fructose-fructose linkages of inulin. The sequences of a number of endo-inulinases are known in the art and are readily obtained, e.g., from keyword or homology searches of publicly available databases, e.g., the NCBI or UNIPROT databases. Endo-inulinases are produced by a number of species. In some embodiments of any of the aspects, the endo-inulinase is a yeast endo-inulinase.

In some embodiments of any of the aspects, the endo-inulinase is an endo-inulinase obtained or purified from an Aspergillus or Penicillium species, or having the sequence of an endo-inulinase of an Aspergillus or Penicillium species. In some embodiments of any of the aspects, the endo-inulinase is an endo-inulinase obtained or purified from an Aspergillus species, or having the sequence of an endo-inulinase of an Aspergillus species. In some embodiments of any of the aspects, the endo-inulinase is an endo-inulinase obtained or purified from Aspergillus niger, or having the sequence of an endo-inulinase of Aspergillus niger. These sequences are known in the art. For example, endo-inulinase sequences are provided in Table 1 for Aspergillus niger and a Penicillium spp. In some embodiments of any of the aspects, the endo-inulinase has at least 80%, at least 85%, at least 90%, at least 95%, at least 98%, or greater sequence identity with one or more of SEQ ID NOs: 1-2. In some embodiments of any of the aspects, the endo-inulinase has at least 95% sequence identity with one or more of SEQ ID NOs: 1-2 and retains the wild-type activity of one or more of SEQ ID NOs: 1-2. Sequences for orthologs found in additional species are readily obtained, e.g., by clicking on the ortholog option for any of the NCBI gene entries in Table 1 or conducting a BLAST search using one of the sequences of Table 1.

TABLE 1
Exemplary endo-inulinases

| Species | Database ID/Accession No. | SEQ ID NO: |
|---|---|---|
| Aspergillus niger | ABB59681.1 | 1 |
| Penicillium sp. TN-88 | BAB19132.1 | 2 |

In some embodiments of any of the aspects, the at least one fructan-degrading enzyme comprises, consists of, or consists essentially of one or more endo-levanases. As used herein “endo-levanase” refers to an enzyme that performs endohydrolysis of the 2→6 beta-D fructose-fructose linkages of levans. The sequences of a number of endo-levanases are known in the art and are readily obtained, e.g., from keyword or homology searches of publicly available databases, e.g., the NCBI or UNIPROT databases. Endo-leavanase are produced by a number of species. In some embodiments of any of the aspects, the endo-inulinase is a bacterial endo-levanase.

In some embodiments of any of the aspects, the endo-levanase is an endo-leavanase obtained or purified from a Bacillus species, or having the sequence of an endo-levanase of a Bacillus species. In some embodiments of any of the aspects, the endo-levanase is an endo-levanase obtained or purified from a Bacillus species, or having the sequence of an endo-levanase of a Bacillus species. In some embodiments of any of the aspects, the endo-levanase is an endo-levanase obtained or purified from Bacillus spizizenii, or having the sequence of an endo-levanase of Bacillus spizizenii. These sequences are known in the art. For example, endo-levanases sequences are provided in Table 14 for Bacillus spizizenii. In some embodiments of any of the aspects, the endo-levanase has at least 80%, at least 85%, at least 90%, at least 95%, at least 98%, or greater sequence identity with SEQ ID NO: 49. In some embodiments of any of the aspects, the endo-levanase has at least 95% sequence identity with SEQ ID NO: 49 and retains the wild-type activity of SEQ ID NO: 49. Sequences for orthologs found in additional species are readily obtained, e.g., by clicking on the ortholog option for any of the NCBI gene entries in Table 14 or conducting a BLAST search using one of the sequences of Table 14.

TABLE 14
Exemplary endo-levanases

| | Database ID/ | SEQ |
|---|---|---|
| Species | Accession No. | ID NO: |
| Bacillus spizizenii | GenBank: SPU08719.1 | 49 |

As used herein “exo-inulinase” refers to an enzyme that performs hydrolysis of the terminal fructose-fructose linkages and/or glucose-fructose linkages of inulin. The sequences of a number of exo-inulinases are known in the art and are readily obtained, e.g., from keyword or homology searches of publicly available databases, e.g., the NCBI or UNIPROT databases. Exo-inulinases are produced by a number of species. In some embodiments of any of the aspects, the exo-inulinase is a yeast exo-inulinase.

In some embodiments of any of the aspects, the at least one fructan-degrading enzyme comprises, consists of, or consists essentially of one or more exo-inulinases which does not comprise endo-inulinase activity. In some embodiments of any of the aspects, the endo-inulinase activity is activity according to EC 3.2.1.7. In some embodiments of any of the aspects, the at least one fructan-degrading enzyme comprises, consists of, or consists essentially of one or more exo-inulinases which also comprise fructosyltransferase activity. In some embodiments of any of the aspects, the at least one fructan-degrading enzyme comprises, consists of, or consists essentially of one or more exo-inulinases which also comprise beta-fructofuranosidase activity. In some embodiments of any of the aspects, the at least one fructan-degrading enzyme comprises, consists of, or consists essentially of one or more exo-inulinases which does not comprise endo-inulinase activity and does comprise fructosyltransferase and/or beta-fructo-furanoside activity.

In some embodiments of any of the aspects, the exo-inulinase is an exo-inulinase obtained or purified from an Aspergillus, Lactobacillus, Paenibacillus, Penicillium, Geobacillus, or Kluyveromyces spp, or having the sequence of an exo-inulinase of an Aspergillus, Lactobacillus, Paenibacillus, Penicillium, Geobacillus, or Kluyveromyces spp. In some embodiments of any of the aspects, the exo-inulinase is an exo-inulinase obtained or purified from an Aspergillus or Kluyveromyces spp, or having the sequence of an exo-inulinase of an Aspergillus or Kluyveromyces spp. In some embodiments of any of the aspects, the exo-inulinase is an exo-inulinase obtained or purified from Aspergillus niger, Aspergillus oryzae, Aspergillus luchuensis, Paenibacillus polymyxa, Penicillium citrinum, Geobacillus stearothermophilus, or Kluyveromyces marxianus (e.g., DMKU3-1042 or CBS6014, see, e.g., US 2020/0205425), or having the sequence of an exo-inulinase of Aspergillus niger, Aspergillus oryzae, Aspergillus luchuensis, Paenibacillus polymyxa, Penicillium citrinum, Geobacillus stearothermophilus, or Kluyveromyces marxianus. In some embodiments of any of the aspects, the exo-inulinase is an exo-inulinase obtained or purified from Aspergillus niger, Aspergillus oryzae, Aspergillus luchuensis, or Kluyveromyces marxianus (e.g., DMKU3-1042 or CBS6014, see, e.g., US 2020/0205425), or having the sequence of an exo-inulinase of Aspergillus niger, Aspergillus oryzae, Aspergillus luchuensis, or Kluyveromyces marxianus. In some embodiments of any of the aspects, the exo-inulinase is an exo-inulinase obtained or purified from Aspergillus oryzae, Aspergillus luchuensis, Paenibacillus polymyxa, Penicillium citrinum, Geobacillus stearothermophilus, or Kluyveromyces marxianus (e.g., DMKU3-1042 or CBS6014, see, e.g., US 2020/0205425), or having the sequence of an exo-inulinase of Aspergillus oryzae, Aspergillus luchuensis, Paenibacillus polymyxa, Penicillium citrinum, Geobacillus stearothermophilus, or Kluyveromyces marxianus. In some embodiments of any of the aspects, the exo-inulinase is an exo-inulinase obtained or purified from Aspergillus oryzae, Aspergillus luchuensis, or Kluyveromyces marxianus (e.g., DMKU3-1042 or CBS6014, see, e.g., US 2020/0205425), or having the sequence of an exo-inulinase of Aspergillus oryzae, Aspergillus luchuensis, or Kluyveromyces marxianus. In some embodiments of any of the aspects, the exo-inulinase is an exo-inulinase obtained or purified from Aspergillus oryzae or having the sequence of an exo-inulinase of Aspergillus oryzae. These sequences are known in the art. For example, exo-inulinase sequences are provided in Table 2 for, e.g., Aspergillus niger, Aspergillus oryzae, Aspergillus luchuensis, and Kluyveromyces marxianus. In some embodiments of any of the aspects, the exo-inulinase has at least 80%, at least 85%, at least 90%, at least 95%, at least 98%, or greater sequence identity with one or more of SEQ ID NOs: 3-7, 29-31, and 42-48. In some embodiments of any of the aspects, the exo-inulinase has at least 95% sequence identity with one or more of SEQ ID NOs: 3-7, 29-31, and 42-48 and retains the wild-type activity of one or more of SEQ ID NOs: 3-7, 29-31, and 42-48. Sequences for orthologs found in additional species are readily obtained, e.g., by clicking on the ortholog option for any of the NCBI gene entries in Table 2 or conducting a BLAST search using one of the sequences of Table 2. Exemplary embodiments of commercially available exo-inulinases can include Catalog No. NATE-1245 from Creative Enzymes (Shirley, NY) and Catalog No. E-EXOIAN from Megazyme (Bray, Ireland). Further exemplary exo-inulinases are described in Goh et al (2007) Appl. Environ. Microbiol. 73 (18) 5716-5724; Müller, M. and Seyfarth, W. (1997). New Phytol. 136, 89-96; Paludan-Müller, et al. (2002). System. Appl. Microbiol. 25, 13-20; US patent publication US 2019/0174773; and International Patent Publication WO 2010/097416; each of which is incorporated by reference herein in its entirety.

In some embodiments of any of the aspects, the exo-inulinase has at least 80%, at least 85%, at least 90%, at least 95%, at least 98%, or 100% sequence identity with SEQ ID NO: 3. In some embodiments of any of the aspects, the exo-inulinase has at least 95% sequence identity with SEQ ID NO: 3 and retains the wild-type activity of SEQ ID NO: 3. In some embodiments of any of the aspects, the exo-inulinase has at least 80%, at least 85%, at least 90%, at least 95%, at least 98%, or 100% sequence identity with SEQ ID NO: 4. In some embodiments of any of the aspects, the exo-inulinase has at least 95% sequence identity with SEQ ID NO: 4 and retains the wild-type activity of SEQ ID NO: 4. In some embodiments of any of the aspects, the exo-inulinase has at least 80%, at least 85%, at least 90%, at least 95%, at least 98%, or 100% sequence identity with SEQ ID NO: 5. In some embodiments of any of the aspects, the exo-inulinase has at least 95% sequence identity with SEQ ID NO: 5 and retains the wild-type activity of SEQ ID NO: 5. In some embodiments of any of the aspects, the exo-inulinase has at least 80%, at least 85%, at least 90%, at least 95%, at least 98%, or 100% sequence identity with SEQ ID NO: 6. In some embodiments of any of the aspects, the exo-inulinase has at least 95% sequence identity with SEQ ID NO: 6 and retains the wild-type activity of SEQ ID NO: 6. In some embodiments of any of the aspects, the exo-inulinase has at least 80%, at least 85%, at least 90%, at least 95%, at least 98%, or 100% sequence identity with SEQ ID NO: 7. In some embodiments of any of the aspects, the exo-inulinase has at least 95% sequence identity with SEQ ID NO: 7 and retains the wild-type activity of SEQ ID NO: 7. In some embodiments of any of the aspects, the exo-inulinase has at least 80%, at least 85%, at least 90%, at least 95%, at least 98%, or 100% sequence identity with SEQ ID NO: 29. In some embodiments of any of the aspects, the exo-inulinase has at least 95% sequence identity with SEQ ID NO: 29 and retains the wild-type activity of SEQ ID NO: 29. In some embodiments of any of the aspects, the exo-inulinase has at least 80%, at least 85%, at least 90%, at least 95%, at least 98%, or 100% sequence identity with SEQ ID NO: 30. In some embodiments of any of the aspects, the exo-inulinase has at least 95% sequence identity with SEQ ID NO: 30 and retains the wild-type activity of SEQ ID NO: 30. In some embodiments of any of the aspects, the exo-inulinase has at least 80%, at least 85%, at least 90%, at least 95%, at least 98%, or 100% sequence identity with SEQ ID NO: 31. In some embodiments of any of the aspects, the exo-inulinase has at least 95% sequence identity with SEQ ID NO: 31 and retains the wild-type activity of SEQ ID NO: 31. In some embodiments of any of the aspects, the exo-inulinase has at least 80%, at least 85%, at least 90%, at least 95%, at least 98%, or 100% sequence identity with SEQ ID NO: 42. In some embodiments of any of the aspects, the exo-inulinase has at least 95% sequence identity with SEQ ID NO: 42 and retains the wild-type activity of SEQ ID NO: 42. In some embodiments of any of the aspects, the exo-inulinase has at least 80%, at least 85%, at least 90%, at least 95%, at least 98%, or 100% sequence identity with SEQ ID NO: 43. In some embodiments of any of the aspects, the exo-inulinase has at least 95% sequence identity with SEQ ID NO: 43 and retains the wild-type activity of SEQ ID NO: 43. In some embodiments of any of the aspects, the exo-inulinase has at least 80%, at least 85%, at least 90%, at least 95%, at least 98%, or 100% sequence identity with SEQ ID NO: 44. In some embodiments of any of the aspects, the exo-inulinase has at least 95% sequence identity with SEQ ID NO: 44 and retains the wild-type activity of SEQ ID NO: 44. In some embodiments of any of the aspects, the exo-inulinase has at least 80%, at least 85%, at least 90%, at least 95%, at least 98%, or 100% sequence identity with SEQ ID NO: 45. In some embodiments of any of the aspects, the exo-inulinase has at least 95% sequence identity with SEQ ID NO: 45 and retains the wild-type activity of SEQ ID NO: 45. In some embodiments of any of the aspects, the exo-inulinase has at least 80%, at least 85%, at least 90%, at least 95%, at least 98%, or 100% sequence identity with SEQ ID NO: 46. In some embodiments of any of the aspects, the exo-inulinase has at least 95% sequence identity with SEQ ID NO: 46 and retains the wild-type activity of SEQ ID NO: 46. In some embodiments of any of the aspects, the exo-inulinase has at least 80%, at least 85%, at least 90%, at least 95%, at least 98%, or 100% sequence identity with SEQ ID NO: 47. In some embodiments of any of the aspects, the exo-inulinase has at least 95% sequence identity with SEQ ID NO: 47 and retains the wild-type activity of SEQ ID NO: 47. In some embodiments of any of the aspects, the exo-inulinase has at least 80%, at least 85%, at least 90%, at least 95%, at least 98%, or 100% sequence identity with SEQ ID NO: 48. In some embodiments of any of the aspects, the exo-inulinase has at least 95% sequence identity with SEQ ID NO: 48 and retains the wild-type activity of SEQ ID NO: 48.

TABLE 2
Exemplary exo-inulinases

| | Database ID/ | SEQ |
|---|---|---|
| Species | Accession No. | ID NO: |
| Aspergillus oryzae | ABY49829 | 3 |
| Aspergillus niger | CAK41278 | 4 |
| Aspergillus oryzae | BAE61799 | 5 |
| Kluyveromyces marxianus | XP_022674031 | 6 |
| MMKU3-1042 | | |
| Lactobacillus crispatus (DSM | | 7 |
| 29598) (see, e.g., US Patent | | |
| Publication 2019/0174773) | | |
| Kluyveromyces marxianus | AF178979.1 | 29 |
| Aspergillus luchuensis | GAT18742.1 | 30 |
| Aspergillus luchuensis | GAA87698.1 | 31 |
| synthetic construct derived from | AVH78393.1 | 42 |
| Talaromyces purpureogenus, | | |
| optimized for expression in | | |
| Eschericia coli | | |
| Aspergillus oryzae | AGI61107.1 | 43 |
| Aspergillus oryzae | AVJ46872.1 | 44 |
| Aspergillus niger | AHC54391.1 | 45 |
| Paenibacillus polymyxa | Genbank: AAL82575.1 | 46 |
| Penicillium citrinum | GenBank: AIY26830.1 | 47 |
| Geobacillus stearothermophilus | GenBank: BAC45010.1 | 48 |

As used herein “exo-levanase” refers to an enzyme that performs hydrolysis of the terminal 2→6 beta-D fructose-fructose linkages of levans. The sequences of a number of exo-levanases are known in the art and are readily obtained, e.g., from keyword or homology searches of publicly available databases, e.g., the NCBI or UNIPROT databases. Exo-levanases are produced by a number of species. In some embodiments of any of the aspects, the exo-levanase is a bacterial exo-levanase.

In some embodiments of any of the aspects, the at least one fructan-degrading enzyme comprises, consists of, or consists essentially of one or more exo-levanase which does not comprise endo-levanase activity. In some embodiments of any of the aspects, the at least one fructan-degrading enzyme comprises, consists of, or consists essentially of one or more exo-levanase which does not comprise endo-levanase activity or endo-inulinase activity. In some embodiments of any of the aspects, the at least one fructan-degrading enzyme comprises, consists of, or consists essentially of one or more exo-levanases which also comprise fructosyltransferase activity. In some embodiments of any of the aspects, the at least one fructan-degrading enzyme comprises, consists of, or consists essentially of one or more exo-levanases which also comprise beta-fructofuranosidase activity. In some embodiments of any of the aspects, the at least one fructan-degrading enzyme comprises, consists of, or consists essentially of one or more exo-levanase which does not comprise endo-inulinase activity, does not comprise endo-levanases activity, and does comprise fructosyltransferase and/or beta-fructo-furanoside activity.

In some embodiments of any of the aspects, the exo-levanase is an exo-levanase obtained or purified from a Gluconacetobacter spp, or having the sequence of an exo-levanase of a Gluconacetobacter spp. In some embodiments of any of the aspects, the exo-levanase is an exo-levanase obtained or purified from Gluconacetobacter johannae, or having the sequence of an exo-levanase of Gluconacetobacter johannae. These sequences are known in the art. For example, exo-levanse sequences are provided in Table 15 for, e.g., Gluconacetobacter johannae. In some embodiments of any of the aspects, the exo-levanase has at least 80%, at least 85%, at least 90%, at least 95%, at least 98%, or greater sequence identity with SEQ ID NO: 50. In some embodiments of any of the aspects, the exo-levanase has at least 95% sequence identity with SEQ ID NO: 50 and retains the wild-type activity of SEQ ID NO: 50. Sequences for orthologs found in additional species are readily obtained, e.g., by clicking on the ortholog option for any of the NCBI gene entries in Table 15 or conducting a BLAST search using one of the sequences of Table 15.

TABLE 15
Exemplary exo-levanase

| | Database ID/ | SEQ |
|---|---|---|
| Species | Accession No. | ID NO: |
| Gluconacetobacter johannae | QEP49755.1 | 50 |

In some embodiments of any of the aspects, the at least one fructan-degrading enzyme comprises, consists of, or consists essentially of one or more of: at least one exo-inulinase, at least one exo-levanase; at least one endo-levanase, and at least one endo-inulinase. In some embodiments of any of the aspects, the at least one fructan-degrading enzyme comprises, consists of, or consists essentially of: at least one exo-inulinase, at least one exo-levanase; at least one endo-levanase, and at least one endo-inulinase. In some embodiments of any of the aspects, the at least one fructan-degrading enzyme comprises, consists of, or consists essentially of: at least one endo-levanase and at least one endo-inulinase.

In some embodiments of any of the aspects, the at least one fructan-degrading enzyme comprises, consists of, or consists essentially of both at least one exo-inulinase and at least one endo-inulinase. In some embodiments of any of the aspects, the composition or combination does not comprise an inulinase obtained or purified from Aspergillus niger, or having the sequence of an inulinase of Aspergillus niger. In some embodiments of any of the aspects, the composition or combination does not comprise an exo-inulinase obtained or purified from Aspergillus niger, or having the sequence of an exo-inulinase of Aspergillus niger. In some embodiments of any of the aspects, the composition or combination does not comprise an endo-inulinase obtained or purified from Aspergillus niger, or having the sequence of an exo-inulinase of Aspergillus niger.

As used herein “amyloglucosidase” refers to an enzyme that performs hydrolysis of an alpha-glycosidic linkage to release a terminal glucose. The sequences of a number of amyloglucosidases are known in the art and are readily obtained, e.g., from keyword or homology searches of publicly available databases, e.g., the NCBI or UNIPROT databases. Amyloglucosidases are produced by a number of species. In some embodiments of any of the aspects, the amyloglucosidase is a yeast amyloglucosidase.

In some embodiments of any of the aspects, the amyloglucosidase is an amyloglucosidase obtained or purified from an Aspergillus or Saccharomyces spp, or having the sequence of an amyloglucosidase of an Aspergillus or Saccharomyces spp. In some embodiments of any of the aspects, the amyloglucosidase is an amyloglucosidase obtained or purified from Aspergillus niger, or having the sequence of an amyloglucosidase of Aspergillus niger. These sequences are known in the art. For example, amyloglucosidase sequences are provided in Table 3 for Aspergillus fumigatus, Saccharomyces cerevisiae, and Aspergillus niger. In some embodiments of any of the aspects, the amyloglucosidase has at least 80%, at least 85%, at least 90%, at least 95%, at least 98%, or greater sequence identity with one or more of SEQ ID NOs: 8-10. In some embodiments of any of the aspects, the amyloglucosidase has at least 95% sequence identity with one or more of SEQ ID NOs: 8-10 and retains the wild-type activity of one or more of SEQ ID NOs: 8-10. Sequences for orthologs found in additional species are readily obtained, e.g., by clicking on the ortholog option for any of the NCBI gene entries in Table 3 or conducting a BLAST search using one of the sequences of Table 3.

TABLE 3
Exemplary amyloglucosidase

| | Database ID/ | SEQ |
|---|---|---|
| Species | Accession No. | ID NO: |
| Aspergillus fumigatus Af293 | 3507868 | 8 |
| Saccharomyces cerevisiae S288C | 856314 | 9 |
| Aspergillus niger | GAQ33831 | 10 |

In some embodiments of any of the aspects, the at least one fructan-degrading enzyme comprises, consists of, or consists essentially of at least one exo-inulinase, at least one endo-inulinase, and at least one amyloglucosidase. In some embodiments of any of the aspects, the at least one fructan-degrading enzyme comprises, consists of, or consists essentially of at least one exo-inulinase and at least one amyloglucosidase. In some embodiments of any of the aspects, the at least one fructan-degrading enzyme comprises, consists of, or consists essentially of at least one endo-inulinase and at least one amyloglucosidase.

In some embodiments of any of the aspects, the at least one fructan-degrading enzyme comprises, consists of, or consists essentially of a) at least one exo-inulinase and/or exo-levanase, b) at least one endo-inulinase and/or exo-levanase, and c) at least one amyloglucosidase. In some embodiments of any of the aspects, the at least one fructan-degrading enzyme comprises, consists of, or consists essentially of a) at least one exo-inulinase and/or exo-levanase; and b) at least one amyloglucosidase. In some embodiments of any of the aspects, the at least one fructan-degrading enzyme comprises, consists of, or consists essentially of a) at least one endo-inulinase and/or endo-levanase, and b) at least one amyloglucosidase.

In some embodiments of any of the aspects, the at least one fructan-degrading enzyme does not comprise an amyloglucosidase. In some embodiments of any of the aspects, the at least one fructan-degrading enzyme does not comprise an enzyme with amyloglucosidase activity. In some embodiments of any of the aspects, the composition or combination provided herein does not comprise an amyloglucosidase. In some embodiments of any of the aspects, the composition or combination provided herein does not comprise an enzyme with amyloglucosidase activity. In some embodiments of any of the aspects, the composition or combination provided herein does not comprise an exogenous amyloglucosidase, e.g., an amyloglucosidase that was not present or provided as part of the biological material of a composition or combination element such as a yeast or a food.

In some embodiments of any of the aspects, the at least one fructan-degrading enzyme does not comprise an endo-levanase or an enzyme with endo-levanase activity. In some embodiments of any of the aspects, the composition or combination does not comprise an endo-levanase or an enzyme with endo-levanase activity. In some embodiments of any of the aspects, the at least one fructan-degrading enzyme does not comprise an exo-levanase or an enzyme with exo-levanase activity. In some embodiments of any of the aspects, the composition or combination does not comprise an exo-levanase or an enzyme with exo-levanase activity. In some embodiments of any of the aspects, the at least one fructan-degrading enzyme does not comprise an exo-levanase, an endo-levanase, or an enzyme with either endo- or exo-levanase activity. In some embodiments of any of the aspects, the composition or combination does not comprise an exo-levanase, an endo-levanase, or an enzyme with either endo- or exo-levanase activity.

In some embodiments of any of the aspects, the inulinase has at least 80%, at least 85%, at least 90%, at least 95%, at least 98%, or 100% sequence identity with any one of SEQ ID NOs: 305-799. In some embodiments of any of the aspects, the inulinase has at least 95% sequence identity with any one of SEQ ID NOs: 305-799. In some embodiments of any of the aspects, the inulinase has at at least 95% sequence identity with any one of SEQ ID NOs: 305-799 and retains the wild-type activity of the reference sequence selected from SEQ ID NOs: 305-799. In some embodiments of any of the aspects, the inulinase has the sequence of any one of SEQ ID NOs: 305-799.

Galactosidases include alpha-galactosidases and beta-galactosidases. In some embodiments of any of the aspects, the at least one galactosidase comprises, consists of, or consists essentially of at least one alpha-galactosidase and at least one beta-galactosidase. In some embodiments of any of the aspects, the at least one galactosidase comprises, consists of, or consists essentially of an alpha-galactosidase. In some embodiments of any of the aspects, the at least one galactosidase comprises, consists of, or consists essentially of a beta-galactosidase.

As used herein, “alpha-galactosidase” refers to an enzyme that hydrolyses terminal alpha-galactosyl moieties from oligosaccharides, glycolipids, and/or glycoproteins. The sequences of a number of alpha-galactosidases are known in the art and are readily obtained, e.g., from keyword or homology searches of publicly available databases, e.g., the NCBI or UNIPROT databases. Alpha-galactosidases are produced by a number of species. In some embodiments of any of the aspects, the alpha-galactosidase is a yeast alpha-galactosidase.

In some embodiments of any of the aspects, the alpha-galactosidase is an alpha-galactosidase obtained or purified from an Aspergillus species, or having the sequence of an alpha-galactosidase of an Aspergillus species. These sequences are known in the art. For example, alpha-galactosidase sequences are provided in Table 4 for Aspergillus niger. In some embodiments of any of the aspects, the alpha-galactosidase has at least 80%, at least 85%, at least 90%, at least 95%, at least 98%, or greater sequence identity with one or more of SEQ ID NOs: 11-14. In some embodiments of any of the aspects, the alpha-galactosidase has at least 95% sequence identity with one or more of SEQ ID NOs: 11-14 and retains the wild-type activity of one or more of SEQ ID NOs: 11-14. Sequences for orthologs found in additional species are readily obtained, e.g., by clicking on the ortholog option for any of the NCBI gene entries in Table 4 or conducting a BLAST search using one of the sequences of Table 4.

TABLE 4
Exemplary alpha-galactosidases

| | Database ID/ | SEQ |
|---|---|---|
| Species | Accession No. | ID NO: |
| Aspergillus niger CBS 101883 | 37101527 | 11 |
| Aspergillus niger CBS 101883 | 37099996 | 12 |
| Aspergillus niger | CAK44692 | 13 |
| Aspergillus niger (see, e.g., | | 14 |
| U.S. Pat. No. 6,197,566) | | |

As used herein, “beta-galactosidase” refers to an enzyme that hydrolyses terminal beta-galactosyl moieties from oligosaccharides, glycolipids, and/or glycoproteins, most notably from lactose. The sequences of a number of beta-galactosidases are known in the art and are readily obtained, e.g., from keyword or homology searches of publicly available databases, e.g., the NCBI or UNIPROT databases. Beta-galactosidases are produced by a number of species. In some embodiments of any of the aspects, the beta-galactosidase is a yeast beta-galactosidase.

In some embodiments of any of the aspects, the beta-galactosidase is a beta-galactosidase obtained or purified from an Aspergillus species, or having the sequence of a beta-galactosidase of an Aspergillus species. In some embodiments of any of the aspects, the beta-galactosidase is a beta-galactosidase obtained or purified from Aspergillus niger, or having the sequence of a beta-galactosidase of Aspergillus niger. These sequences are known in the art. For example, an exemplary beta-galactosidase sequence is provided in Table 5 for Aspergillus niger. In some embodiments of any of the aspects, the beta-galactosidase has at least 80%, at least 85%, at least 90%, at least 95%, at least 98%, or greater sequence identity with SEQ ID NO: 15. In some embodiments of any of the aspects, the beta-galactosidase has at least 95% sequence identity with SEQ ID NO: 15 and retains the wild-type activity of SEQ ID NO: 15. Sequences for orthologs found in additional species are readily obtained, e.g., by clicking on the ortholog option for any of the NCBI gene entries in Table 5 or conducting a BLAST search using one of the sequences of Table 5.

TABLE 5
Exemplary beta-galactosidases

| | Database ID/ | SEQ |
|---|---|---|
| Species | Accession No. | ID NO: |
| Aspergillus niger | BGALA_ASPOR | 15 |

Alpha and beta galactosidases are also described, e.g., in U.S. Pat. Nos. 9,402,885 and 6,197,566; which are incorporated by reference herein in their entireties.

In some embodiments of any of the aspects, the beta galactosidase is a lactase. As used herein, “lactase” refers to any enzyme that converts lactose to galactose and glucose monomers. The sequences of a number of lactases are known in the art and are readily obtained, e.g., from keyword or homology searches of publicly available databases, e.g., the NCBI or UNIPROT databases. Lactases are produced by a number of species. In some embodiments of any of the aspects, the lactase is a yeast lactase. In some embodiments of any of the aspects, the lactase is a fungal lactacase. In some embodiments of any of the aspects, the lactase is a mammalian (e.g., human) lactase.

In some embodiments of any of the aspects, the lactase is a lactase obtained or purified from a Kluyveromyces or Aspergillus species, or having the sequence of a lactase of a Kluyveromyces or Aspergillus species. In some embodiments of any of the aspects, the lactase is a lactase obtained or purified from Kluyveromyces lactis, Kluyveromyces marxianus, Aspergillus niger, or Aspergillus oryzae, or having the sequence of a lactase of Kluyveromyces lactis, Kluyveromyces marxianus, Aspergillus niger, or Aspergillus oryzae. These sequences are known in the art. For example, exemplary lactase sequences are provided in Table 13. In some embodiments of any of the aspects, the lactase has at least 80%, at least 85%, at least 90%, at least 95%, at least 98%, or greater sequence identity with one of SEQ ID NOs: 36-41. In some embodiments of any of the aspects, the lactase has at least 95% sequence identity with one of SEQ ID NOs: 36-41 and retains the wild-type activity of one of SEQ ID NOs: 36-41. Sequences for orthologs found in additional species are readily obtained, e.g., by clicking on the ortholog option for any of the NCBI gene entries in Table 13 or conducting a BLAST search using one of the sequences of Table 13.

TABLE 13
Exemplary lactases

| | Database ID/ | SEQ |
|---|---|---|
| Species | Accession No. | ID NO: |
| Kluvyveromyces lactis | PSP: P00723.2 | 36 |
| Kluyveromyces marxianus (anamorph is | KAG0680849.1 | 37 |
| Candida pseudotropicalis) | | |
| Aspergillus niger | EHA26981.1 | 38 |
| Aspergillus niger | EHA26848.1 | 39 |
| Aspergillus oryzae | AHI44625.1 | 40 |
| Homo sapiens preprotein (mature | NP_002290.2 | 41 |
| protein is aa 869-1927) | | |

As used herein, “sorbitol-degrading enzyme” refers to any enzyme that catabolizes sorbitol or a catabolic product of sorbitol. Sorbitol is a sugar alcohol having the structure of Formula I. Sorbitol-degrading enzymes include sorbitol dehydrogenase and sorbitol oxidase.

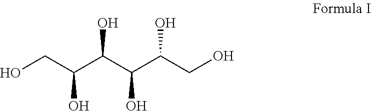 [SMILES: OC[C@@H](O)[C@@H](O)[C@H](O)[C@@H](O)CO]

Sorbitol is catabolized to fructose by sorbitol dehydrogenases, e.g., sorbitol-6-phosphate-2-dehydrogenase. In some embodiments of any of the aspects, the sorbitol-degrading enzyme is a sorbitol dehydrogenase. In some embodiments of any of the aspects, a composition or combination comprising sorbitol dehydrogenase further comprises NAD+, which is a sorbitol dehydrogenase cofactor.

The sequences of a number of sorbitol dehydrogenases are known in the art and are readily obtained, e.g., from keyword or homology searches of publicly available databases, e.g., the NCBI or UNIPROT databases. Sorbitol dehydrogenases are produced by a number of species. In some embodiments of any of the aspects, the sorbitol dehydrogenase is a mammalian sorbitol dehydrogenase.

In some embodiments of any of the aspects, the sorbitol dehydrogenase is a sorbitol dehydrogenase obtained or purified from a Ovis or Rattus species, or having the sequence of a sorbitol dehydrogenase of a Ovis or Rattus species. In some embodiments of any of the aspects, the sorbitol dehydrogenase is a sorbitol dehydrogenase obtained or purified from a sheep or a rat, or having the sequence of a sorbitol dehydrogenase of a sheep or a rat. These sequences are known in the art. For example, sorbitol dehydrogenase sequences are provided in Table 6 for human, sheep, and rat. In some embodiments of any of the aspects, the sorbitol dehydrogenase has at least 80%, at least 85%, at least 90%, at least 95%, at least 98%, or greater sequence identity with one or more of SEQ ID NOs: 16-18. In some embodiments of any of the aspects, the sorbitol dehydrogenase has at least 95% sequence identity with one or more of SEQ ID NOs: 16-18 and retains the wild-type activity of one or more of SEQ ID NOs: 16-18. Sequences for orthologs found in additional species are readily obtained, e.g., by clicking on the ortholog option for any of the NCBI gene entries in Table 6 or conducting a BLAST search using one of the sequences of Table 6.

TABLE 6
Exemplary sorbitol dehydrogenases

| Species | NCBI ID | SEQ ID NO: |
|---|---|---|
| Homo sapiens | 6652 | 16 |
| Rattus norvegicus | 24788 | 17 |
| Ovis aries | 101115280 | 18 |

In some embodiments of any of the aspects, the sorbitol-degrading enzyme is a sorbitol oxidase. A sorbitol oxidase is an enzyme that catalyzes the oxidation of sorbitol to glucose. O₂ is a cofactor for sorbitol oxidase. The sequences of a number of sorbitol oxidases are known in the art and are readily obtained, e.g., from keyword or homology searches of publicly available databases, e.g., the NCBI or UNIPROT databases. Sorbitol oxidases are produced by a number of species. In some embodiments of any of the aspects, the sorbitol oxidase is a yeast sorbitol oxidase.

In some embodiments of any of the aspects, the sorbitol oxidase is a sorbitol oxidase obtained or purified from a Streptomyces species, or having the sequence of a sorbitol oxidase of a Streptomyces species. In some embodiments of any of the aspects, the sorbitol oxidase is a sorbitol oxidase obtained or purified from Streptomyces griseoflavus, or having the sequence of a sorbitol oxidase of Streptomyces griseoflavus. These sequences are known in the art. For example, sorbitol oxidase sequences are provided in Table 7 for Streptomyces griseoflavus. In some embodiments of any of the aspects, the sorbitol oxidase has at least 80%, at least 85%, at least 90%, at least 95%, at least 98%, or greater sequence identity with SEQ ID NO: 19. In some embodiments of any of the aspects, the sorbitol oxidase has at least 95% sequence identity with SEQ ID NO: 19 and retains the wild-type activity of SEQ ID NO: 19. Sequences for orthologs found in additional species are readily obtained, e.g., by clicking on the ortholog option for any of the NCBI gene entries in Table 7 or conducting a BLAST search using one of the sequences of Table 7.

TABLE 7
Exemplary sorbitol oxidase

| | Database ID/ | SEQ |
|---|---|---|
| Species | Accession No. | ID NO: |
| Streptomyces griseoflavus Tu4000 | EFL38250.1 | 19 |

As used herein, “mannitol-degrading enzyme” refers to any enzyme that catabolizes mannitol or a catabolic product of mannitol. Mannitol is a sugar alcohol having the structure of Formula II. Mannitol-degrading enzymes include mannitol dehydrogenase and mannitol oxidase.

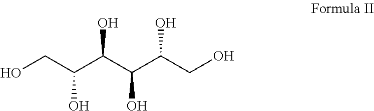 [SMILES: OC[C@@H](O)[C@@H](O)[C@H](O)[C@H](O)CO]

Mannitol is catabolized to mannose by mannitol dehydrogenases. In some embodiments of any of the aspects, the mannitol-degrading enzyme is a mannitol dehydrogenase. In some embodiments of any of the aspects, a composition or combination comprising mannitol dehydrogenase further comprises NAD+, which is a mannitol dehydrogenase cofactor.

The sequences of a number of mannitol dehydrogenases are known in the art and are readily obtained, e.g., from keyword or homology searches of publicly available databases, e.g., the NCBI or UNIPROT databases. Mannitol dehydrogenases are produced by a number of species. In some embodiments of any of the aspects, the mannitol dehydrogenase is a yeast or bacterial mannitol dehydrogenase. In some embodiments of any of the aspects, the mannitol dehydrogenase is a bacterial mannitol dehydrogenase.

In some embodiments of any of the aspects, the mannitol dehydrogenase is a mannitol dehydrogenase obtained or purified from a Saccharomyces, Pseudomas, Escherichia, Leuconostoc, or Gluconobacter spp., or having the sequence of a mannitol dehydrogenase of a Saccharomyces, Pseudomas, Escherichia, Leuconostoc, or Gluconobacter spp. In some embodiments of any of the aspects, the mannitol dehydrogenase is a mannitol dehydrogenase obtained or purified from S. cerevisiae, P. fluorescens, E. coli, L. mesenteroides, or G. oxydans, or having the sequence of a mannitol dehydrogenase of S. cerevisiae, P. fluorescens, E. coli, L. mesenteroides, or G. oxydans. These sequences are known in the art. For example, mannitol dehydrogenase sequences are provided in Table 8 for S. cerevisiae, P. fluorescens, E. coli, L. mesenteroides, or G. oxydans. In some embodiments of any of the aspects, the mannitol dehydrogenase has at least 80%, at least 85%, at least 90%, at least 95%, at least 98%, or greater sequence identity with one or more of SEQ ID NOs: 20-26. In some embodiments of any of the aspects, the mannitol dehydrogenase has at least 95% sequence identity with one or more of SEQ ID NOs: 20-26 and retains the wild-type activity of one or more of SEQ ID NOs: 20-26. Sequences for orthologs found in additional species are readily obtained, e.g., by clicking on the ortholog option for any of the NCBI gene entries in the following Table or conducting a BLAST search using one of the sequences of the following Table.

TABLE 8
Exemplary mannitol dehydrogenases

| | Database ID/ | SEQ |
|---|---|---|
| Species | Accession No. | ID NO: |
| Pseudomonas fluorescens | 57515047 | 20 |
| Escherichia coli | OYI64916.1 | 21 |
| Escherichia coli | ETI79791.1 | 22 |
| Leuconostoc mesenteroides | 222544050 | 23 |
| Leuconostoc mesenteroides | ACT22631 | 24 |
| Saccharomyces cerevisiae S288C | NP_010844 | 25 |
| Gluconobacter oxydans | GEC60258.1 | 26 |

In some embodiments of any of the aspects, the mannitol-degrading enzyme is a mannitol oxidase. A mannitol oxidase is an enzyme that catalyzes the oxidation of mannitol to mannose. O₂ is a cofactor for mannitol oxidase. Mannitol oxidases are produced by a number of species. In some embodiments of any of the aspects, the mannitol oxidase is a yeast mannitol oxidase or a gastropod mannitol oxidase. Further discussion of mannitol oxidases and their isolation can be found, e.g., in Vorhaben J E et al (1986). Int. J. Biochem. 18 (4): 337-44; and Large A T, et al. (1993). Protoplasma. 175 (3-4): 93-101; each of which is incorporated by reference herein in its entirety.

Some oxidases can catabolize both mannitol and sorbitol. Accordingly, in some embodiments, an enzyme identified herein as a “sorbitol oxidase” or a “mannitol oxidase” can comprise both of those activities, e.g., it can be a “mannitol and sorbitol oxidase.” In any of the embodiments of any of the aspects herein, wherein a “sorbitol oxidase” or “mannitol oxidase” is recited, a “mannitol and sorbitol oxidase” is contemplated as meeting the recitation and/or can be used instead. An oxidase can have a preference for either sorbitol or mannitol.

In some embodiments of any of the aspects, mannitol and sorbitol oxidase is a mannitol and sorbitol oxidase obtained or purified from a Streptomyces or Acidothermus species, or having the sequence of a mannitol and sorbitol oxidase of a Streptomyces or Acidothermus species. In some embodiments of any of the aspects, mannitol and sorbitol oxidase is a mannitol and sorbitol oxidase obtained or purified from Streptomyces, Streptomyces sp. H7775, or Acidothermus cellulolyticus, or having the sequence of a mannitol and sorbitol oxidase of Streptomyces, Streptomyces sp. H7775, or Acidothermus cellulolyticus. These sequences are known in the art. For example, exemplary mannitol and sorbitol oxidase sequences are provided in Table 12. In some embodiments of any of the aspects, the mannitol and sorbitol oxidase has at least 80%, at least 85%, at least 90%, at least 95%, at least 98%, or greater sequence identity with at least one of SEQ ID NOs: 32-35. In some embodiments of any of the aspects, the mannitol and sorbitol oxidase has at least 95% sequence identity with at least one of SEQ ID NOs: 32-35 and retains the wild-type activity of at least one of SEQ ID NOs: 32-35. Sequences for orthologs found in additional species are readily obtained, e.g., by clicking on the ortholog option for any of the NCBI gene entries in Table 12 or conducting a BLAST search using one of the sequences of Table 12.

TABLE 12
Exemplary mannitol and sorbitol oxidases

| | Database ID/ | SEQ |
|---|---|---|
| Species | Accession No. | ID NO: |
| Streptomyces sp. | WP_011030685.1 | 32 |
| Acidothermus cellulolyticus | WP_011719559.1 | 33 |
| Streptomyces sp. H7775 | n/a | 34 |
| Streptomyces sp. | BAA95146.1 | 35 |

In some embodiments of any of the aspects, the mannitol and/or sorbitol oxidase has at least 80%, at least 85%, at least 90%, at least 95%, at least 98%, or 100% sequence identity with any one of SEQ ID NOs: 51-304. In some embodiments of any of the aspects, the mannitol and/or sorbitol oxidase has at least 95% sequence identity with any one of SEQ ID NOs: 51-304. In some embodiments of any of the aspects, the mannitol and/or sorbitol oxidase has at at least 95% sequence identity with any one of SEQ ID NOs: 51-304 and retains the wild-type activity of the reference sequence selected from SEQ ID NOs: 51-304. In some embodiments of any of the aspects, the mannitol and/or sorbitol oxidase has the sequence of any one of SEQ ID NOs: 51-304.

As used herein, “glucose isomerase” refers to any enzyme that converts glucose to fructose or fructose to glucose. Glucose isomerase is also known in the art as xylose isomerase. The sequences of a number of glucose isomerases are known in the art and are readily obtained, e.g., from keyword or homology searches of publicly available databases, e.g., the NCBI or UNIPROT databases. Glucose isomerases are produced by a number of species. In some embodiments of any of the aspects, the glucose isomerase is a yeast glucose isomerase.

In some embodiments of any of the aspects, glucose isomerase is a glucose isomerase obtained or purified from a Streptomyces species, or having the sequence of a glucose isomerase of a Streptomyces species. In some embodiments of any of the aspects, glucose isomerase is a glucose isomerase obtained or purified from Streptomyces murinus, or having the sequence of a glucose isomerase of Streptomyces murinus. These sequences are known in the art. For example, a glucose isomerase sequence is provided in Table 9 for Streptomyces murinus. In some embodiments of any of the aspects, the glucose isomerase has at least 80%, at least 85%, at least 90%, at least 95%, at least 98%, or greater sequence identity with SEQ ID NO: 27. In some embodiments of any of the aspects, the glucose isomerase has at least 95% sequence identity with SEQ ID NO: 27 and retains the wild-type activity of SEQ ID NOs: 27. Sequences for orthologs found in additional species are readily obtained, e.g., by clicking on the ortholog option for any of the NCBI gene entries in Table 9 or conducting a BLAST search using one of the sequences of Table 9.

TABLE 9
Exemplary glucose isomerase

| | Database ID/ | SEQ |
|---|---|---|
| Species | Accession No. | ID NO: |
| Streptomyces murinus | CAA00885.1 | 27 |

As used herein, “mannose isomerase” refers to any enzyme that converts mannose to fructose or fructose to mannose. The sequences of a number of mannose isomerases are known in the art and are readily obtained, e.g., from keyword or homology searches of publicly available databases, e.g., the NCBI or UNIPROT databases. Mannose isomerases are produced by a number of species. In some embodiments of any of the aspects, the mannose isomerase is a yeast mannose isomerase.

In some embodiments of any of the aspects, mannose isomerase is a mannose isomerase obtained or purified from a Thermobifida species, or having the sequence of a mannose isomerase of a Thermobifida species. In some embodiments of any of the aspects, mannose isomerase is a mannose isomerase obtained or purified from Thermobifida fusca, or having the sequence of a mannose isomerase of Thermobifida fusca. These sequences are known in the art. For example, a mannose isomerase sequence is provided in Table 10 for Thermobifida fusca. In some embodiments of any of the aspects, the mannose isomerase has at least 80%, at least 85%, at least 90%, at least 95%, at least 98%, or greater sequence identity with SEQ ID NO: 28. In some embodiments of any of the aspects, the mannose isomerase has at least 95% sequence identity with SEQ ID NO: 28 and retains the wild-type activity of SEQ ID NO: 28. Sequences for orthologs found in additional species are readily obtained, e.g., by clicking on the ortholog option for any of the NCBI gene entries in Table 10 or conducting a BLAST search using one of the sequences of Table 10.

TABLE 10
Exemplary mannose isomerase

| | Database ID/ | SEQ |
|---|---|---|
| Species | Accession No. | ID NO: |
| Thermobifidia fusca | BAP46301.1 | 28 |

It is contemplated herein that multiple members of any of the foregoing categories and classifications can be used in the same composition or combination. Any combination or mixture of the foregoing categories and classifications is contemplated herein. Merely by way of example, the following combinations are contemplated herein:
a. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); one or more sorbitol-degrading enzymes (e.g., one or more sorbitol dehydrogenases and/or sorbitol oxidases); one or more mannitol-degrading enzymes (e.g., one or more mannitol dehydrogenases and/or mannitol oxidases); one or more glucose isomerases; and one or more mannose isomerases.
b. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); one or more sorbitol-degrading enzymes (e.g., one or more sorbitol dehydrogenases and/or sorbitol oxidases); one or more mannitol-degrading enzymes (e.g., one or more mannitol dehydrogenases and/or mannitol oxidases); and one or more glucose isomerases.
c. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); one or more sorbitol-degrading enzymes (e.g., one or more sorbitol dehydrogenases and/or sorbitol oxidases); one or more mannitol-degrading enzymes (e.g., one or more mannitol dehydrogenases and/or mannitol oxidases); and one or more mannose isomerases.
d. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); one or more sorbitol-degrading enzymes (e.g., one or more sorbitol dehydrogenases and/or sorbitol oxidases); and one or more mannitol-degrading enzymes (e.g., one or more mannitol dehydrogenases and/or mannitol oxidases.
e. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); and one or more sorbitol-degrading enzymes (e.g., one or more sorbitol dehydrogenases and/or sorbitol oxidases).
f. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); and one or more mannitol-degrading enzymes (e.g. one or more mannitol dehydrogenases and/or mannitol oxidases).
g. One or more fructan-degrading enzymes (e.g., one or more exo-inulinases, one or more endo-inulinases, one or more exo-levanases, one or more endo-levanases, and/or one or more amyloglucosidases); and one or more galactosidases (e.g., one or more alpha-galactosidases and/or one or more beta-galactosidases).
h. One or more fructan-degrading enzymes (e.g., one or more exo-inulinases, one or more endo-inulinases, one or more exo-levanases, one or more endo-levanases, and/or one or more amyloglucosidases); and one or more sorbitol-degrading enzymes (e.g., one or more sorbitol dehydrogenases and/or sorbitol oxidases).
i. One or more fructan-degrading enzymes (e.g., one or more exo-inulinases, one or more endo-inulinases, one or more exo-levanases, one or more endo-levanases, and/or one or more amyloglucosidases); and one or more mannitol-degrading enzymes (e.g., one or more mannitol dehydrogenases and/or mannitol oxidases).
j. One or more fructan-degrading enzymes (e.g., one or more exo-inulinases, one or more endo-inulinases, one or more exo-levanases, one or more endo-levanases, and/or one or more amyloglucosidases); and one or more glucose isomerases.
k. One or more fructan-degrading enzymes (e.g., one or more exo-inulinases, one or more endo-inulinases, one or more exo-levanases, one or more endo-levanases, and/or one or more amyloglucosidases); and one or more mannose isomerases.
l. One or more fructan-degrading enzymes (e.g., one or more exo-inulinases, one or more endo-inulinases, one or more exo-levanases, one or more endo-levanases, and/or one or more amyloglucosidases); one or more glucose isomerases; and one or more mannose isomerases.
m. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); one or more glucose isomerases; and one or more mannose isomerases.
n. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); and one or more mannose isomerases.
o. One or more fructan-degrading enzymes (e.g., one or more exo-inulinases, one or more endo-inulinases, one or more exo-levanases, one or more endo-levanases, and/or one or more amyloglucosidases); one or more galactosidases (e.g., one or more alpha-galactosidases and/or one or more beta-galactosidases); and one or more glucose isomerases.
   In some embodiments of examples a-o of this paragraph, the one or more fructan-degrading enzymes can comprise or be an exo-inulinase. In some embodiments of examples a-o of this paragraph, the one or more fructan-degrading enzymes can comprise or be an endo-inulinase. In some embodiments of examples a-o of this paragraph, the one or more fructan-degrading enzymes can comprise or be an endo-levanase. In some embodiments of examples a-o of this paragraph, the one or more fructan-degrading enzymes can comprise or be an exo-levanase. In some embodiments of examples a-o of this paragraph, the one or more galactosidases can comprise or be an alpha-galactosidase. In some embodiments of examples a-o of this paragraph, the one or more galactosidases can comprise or be a beta-galactosidase. In some embodiments of examples a-o of this paragraph, the one or more galactosidases can comprise or be a lactase. In some embodiments of examples a-o of this paragraph, the one or more sorbitol-degrading enzymes can comprise or be sorbitol dehydrogenase. In some embodiments of examples a-o of this paragraph, the one or more sorbitol-degrading enzymes can comprise or be sorbitol oxidase. In some embodiments of examples a-o of this paragraph, the one or more mannitol-degrading enzymes can comprise or be mannitol dehydrogenase. In some embodiments of examples a-o of this paragraph, the one or more mannitol-degrading enzymes can comprise or be mannitol oxidase.

The compositions and combinations described herein can further comprise one or more of a cofactor; a metal ion; an antacid; an H2 antagonist; a proton pump inhibitor; a pepstatin protease inhibitor; or a protease inhibitor.

In some embodiments of any of the aspects, an enzyme can be a thermo-resistant or acid-resistant variant. Thermo-resistant or acid-resistant variants can be orthologs of an enzyme described herein from a thermophile (e.g., hyperthermophile) or acidophile species, respectively. Such orthologs can be identified by, e.g., a BLAST search of the thermophile or acidophile's sequence data and selecting the most homologous enzyme found therein. Alternatively, the ortholog can be a sequence with at least 60%, at least 70%, at least 80%, at least 85%, at least 90%, at least 95% or more sequence identity to a reference enzyme described herein. Thermophiles and acidophiles are well known in the art, see, e.g., Acetobacter and Thermus species, and their sequence information is publically available. See, e.g., Brumm et al. PLoS One 2015 10:e0138674; Hirose et al. Microbiology Resource Announcements (2020) 9:e00962-20; Illeghems et al. BMC Genomics (2013) 14:526; Jia et al. Front. Bioeng. Biotechnol. (2017) 5:33; and Zhang et al. Int. J. Mol Sci (2016) 17:1355; each of which is incorporated by reference herein in

Additionally, the sequence of any enzyme described herein can be engineered or mutated to be more thermo- or acid-resistant. In some embodiments of any of the aspects, an enzyme described herein has a sequence which is at least 80% identical to a reference sequence provided herein and comprises one or more mutations that make it more thermo- or acid-resistant, while retaining the wild-type activity. In some embodiments of any of the aspects, an enzyme described herein has a sequence which is at least 95% identical to a reference sequence provided herein and comprises one or more mutations that make it more thermo- or acid-resistant, while retaining the wild-type activity.

One of skill in the art is aware of appropriate changes to make to a sequence to obtain such a variant. By way of non-limiting example, substitution of glycine residues with alanine, substitution of alanine residues with proline, substitution of lysine residues with arginine, an increase in ion pairs, a decrease in β-branched residues within helix structures, a decrease in Asn residues available for deamidation, a decrease in cysteine residues, a decrease in glycine residues, an increase in proline residues, a decrease in loop length, all contribute to thermoresistance. Such alterations are described, e.g., in Vielle Microbiol Mol Biol Rev (2001) 65:1-43; Van den Burg et al. PNAS (1998) 95:2056-2060; Sarkar et al. PNAS (2008) 105 (39): 14808-13; Asial et al. Nature Communications (2013) 4:2901; Hoseki et al. Journal of Biochemistry (1999) 126 (5): 951-6; Sayed et al. The Journal of Biological Chemistry (2014) 289 (3): 1675-87; Perl et al. Nature Structural Biology (2000) 7 (5): 380-3; Lehmann et al. Biochimica et Biophysica Acta (BBA)-Protein Structure and Molecular Enzymology (2000) 1543 (2): 408-415; Sauer et al. Biophysical Journal. (2015) 109 (7): 1420-8; Liu et al. Protein Engineering (2013) 16 (1): 19-25; Lee et al. PLOS ONE (2014) 9 (11):e112751; Mansfeld et al. The Journal of Biological Chemistry (1997) 272 (17): 11152-6; Zhang et al. Scientific Reports (2016) 6:33797; each of which are incorporated by reference herein in their entireties. Similar such alterations are described for acid-resistance, e.g., in Liu et al. Biotechnology and Bioeng (2019) 116:833-43; which is incorporated by reference herein in its entirety. As a further non-limiting example, thermoresistant and acid resistant lactases are known in the art, see, e.g., Chiba et al. Journal of Bioscience and Bioengineering 2015 120:263-7 and Macris. Appl Enivron Microbiol 1982 44:1035-8, each of which is incorporated by reference herein in its entirety.

Appropriate variants can also be designed using software available for such purposes, e.g., Rosetta3 (see Richter et al. PLoS ONE (2011) 6:e19230) and iStable 2.0 (see Chen et al. Computational and Structural Biotech J (2020) 18:622-30) (see generally Thiltgen et al. PLoS ONE 2012 7:e46084) or obtained from a commercial source which will prepare such variants, e.g., as CinderBio (cinderbio.com/), SwissAustral (swissaustral.ch/), and Gene & GreenTK (gene-greentk.com/). The foregoing references are incorporated by reference herein in their entireties.

In some embodiments of any of the aspects, the composition or combination described herein can be formulated as a powder, a solution, a pill, a capsule, a concentrate, a tablet, a soft-gel, a thin-film, a liquid, and/or a syrup. Additional suitable formulations include chewable tablets, quick dissolve tablets, effervescent tablets, reconstitutable powders, elixirs, solutions, suspensions, emulsions, multi-layer tablets, bi-layer tablets, gelatin capsules, hard gelatin capsules, caplets, lozenges, chewable lozenges, beads, powders, granules, particles, microparticles, dispersible granules, and combinations thereof.

In some embodiments of any of the aspects, the composition or combination described herein can be formulated with an enteric coating, e.g., a material that dissolves or degrades after passing through the stomach. Such materials are well known in the art and include, but are not limited to, methyl acrylate-methacrylic acid copolymers; cellulose acetate phthalate (CAP); cellulose acetate succinate; hydroxypropyl methyl cellulose phthalate; hydroxypropyl methyl cellulose acetate succinate (hypromellose acetate succinate); polyvinyl acetate phthalate (PVAP); methyl methacrylate-methacrylic acid copolymers; shellac; cellulose acetate trimellitate; sodium alginate; zein; and an enteric coating aqueous solution comprising ethylcellulose, medium chain triglycerides [coconut], oleic acid, sodium alginate, and stearic acid.

The inventor has surprisingly found that the compositions and combinations described exhibit improved performance in the absence of a capsule and/or enteric coating. This is particularly surprising as nearly every digestive enzyme supplement commercially available is provided in a capsule format. Accordingly, specifically contemplated herein are compositions and combinations formulated as powders, tablets, chewables, and other forms lacking a coating and/or capsule, particularly an enteric coating or capsule. In some embodiments of any of the aspects, a composition or combination described herein does not comprise a coating or capsule, e.g., an enteric coating or capsule. In addition, the inventor has found that increased saliva production at the time of administration also provides improved performance for the compositions and combinations described herein. Accordingly, specifically contemplated herein are compositions and combinations in chewable formulations, e.g., chewable tablets, chewable gummies, or other formats.

It is contemplated herein that the enzymes of the compositions and combinations described herein can be active in vitro or in vivo (e.g., in the mouth, the stomach, or the intestines).

The inventor has surprisingly found that the compositions and combinations described herein exhibit improved performance when administered with proteins and/or fats. Accordingly, contemplated herein are kits instructing the user to consume the composition or combination with proteins and/or fats. Alternatively, the composition or combination can further comprise proteins and/or fats to ensure adequate amounts are administered. In some embodiments of any of the aspects, a composition or combination described herein comprises at least 5% protein and/or fat by weight. In some embodiments of any of the aspects, a composition or combination described herein comprises at least 10% protein and/or fat by weight. In some embodiments of any of the aspects, a composition or combination described herein comprises at least 15% protein and/or fat by weight. In some embodiments of any of the aspects, a composition or combination described herein comprises at least 20% protein and/or fat by weight.

In some embodiments of any of the aspects, a composition or combination described herein comprises from about 5% to about 50% protein and/or fat by weight. In some embodiments of any of the aspects, a composition or combination described herein comprises from about 10% to about 40% protein and/or fat by weight. In some embodiments of any of the aspects, a composition or combination described herein comprises from about 20% to about 30% protein and/or fat by weight. In some embodiments of any of the aspects, a composition or combination described herein comprises from 5% to 50% protein and/or fat by weight. In some embodiments of any of the aspects, a composition or combination described herein comprises from 10% to 40% protein and/or fat by weight. In some embodiments of any of the aspects, a composition or combination described herein comprises from 20% to 30% protein and/or fat by weight.

In some embodiments of any of the aspects, the protein and/or fat comprises, consists of, or consists essentially of protein. In some embodiments of any of the aspects, the protein and/or fat comprises, consists of, or consists essentially of fat.

In some embodiments of any of the aspects, the composition or combination further comprises an emulsifier or other inactive ingredients such as sweeteners and/or flavorings, which can be artificial.

In some embodiments of any of the aspects, the composition or combination is provided in a nutraceutical, dietary formulation, and/or dietary supplement. In some embodiments of any of the aspects, a nutraceutical, dietary formulation, and/or dietary supplement can be provided in the form of a shake, meal replacement shake, drink, smoothie, powder, bars, or the like.

As used herein, “nutraceutical” refers to compositions that are useful in both the nutritional and pharmaceutical field of application. Thus, nutraceutical compositions of the present invention may be used as supplements and/or alternatives to food and beverages, and as pharmaceutical formulations which may be solid formulations such as capsules or tablets, or liquid formulations, such as solutions or suspensions. In some embodiments of any of the aspects, nutraceutical compositions may also comprise food and beverages as described herein, as well as supplement compositions, for example dietary supplements.

A dietary formulation can contain all essential amino acids, as well as essential vitamins and minerals to ensure that the recipient is obtaining all necessary nutrients. When the formulation is in the form of a dietary supplement, the formulation can provide about 5% to 60% of total energy expenditure in terms of calories. Use of such a supplement is expected to produce the same beneficial results as described herein for the dietary formulations when combined with a diet. The dietary formulation or supplement can also be made in powder form by increasing the percent total solids of the formula, using procedures well known to those skilled in the art. The concentrate or powder can then be reconstituted for feeding by adding water (tap or deionized-sterilized water).

In some embodiments of any of the aspects, a dietary formulation or supplement can further comprise additional components such as one or more essential fatty acids, a source of carbohydrate, a source of protein, a source of vitamins and minerals, and an emulsifier. The source of carbohydrates can be any simple or complex carbohydrate, e.g., monosaccharides, disaccharides, or oligosaccharides. In one embodiment the source of carbohydrate is at least one of corn starch, dextrose, and glucose. The source of protein can be any protein hydrolysate or peptide mixtures, amino acid mixtures of high biologic values, e.g., meat, milk, egg or soy proteins. The protein hydrolysate can be partially hydrolyzed in nature and include a substantial fraction of variable chain length peptides, e.g., medium or short chain peptides, e.g., di- and tri-peptides, but have less than about 10% free amino acids, more preferably less than about 5% free amino acids. In one embodiment, only the highest biological value proteins are hydrolyzed, e.g., whey, lactalbumin, casein, egg white, egg solids, soy, or delactosed milk solids. In other embodiments, the protein source is lactose-free, and free amino acids are avoided in the formulation. In some embodiments of any of the aspects, a dietary formulation can further comprise a source of vitamins and minerals. For example, vitamins and minerals in accordance with, or approximately, the Recommended Dietary Allowance (RDA), now called the Daily Reference Intake (DRI). The dietary formulations can also contain nutrients not recommended by the DRI, e.g., nucleotides, beta-carotene, carnitine, and taurine. In some embodiments of any of the aspects, a dietary formulation can further comprise an emulsifier or other inactive ingredients such as sweeteners and/or flavorings, which can be artificial. A dietary formulation of the present invention may be in the form of a dietary supplement or used in a feeding regimen. In one embodiment, the dietary formulation is provided in a form suitable for oral administration. The dietary formulation can contain all essential amino acids, as well as essential vitamins and minerals to ensure that the recipient is obtaining all necessary nutrients. When the formulation is in the form of a dietary supplement, the formulation should provide about 5% to 60% of total energy expenditure in terms of calories.

The compositions and/or combinations described herein can be administered in such oral dosage forms as immediate release, controlled release and/or sustained release tablets or capsules, pills, powders, granules, elixirs, tinctures, suspensions, syrups and emulsions. For oral administration in the form of a tablet or capsule (e.g., a gelatin capsule), one or more enzymes can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or β-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents include, e.g., cellulose and/or glycine.

Examples of formulations suitable for oral administration include, but are not limited to one or more enzyme(s) present in, chewable tablets, quick dissolve tablets, effervescent tablets, dissolvable strips, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, health bars, confections, animal feeds, cereals, yogurts, cereal coatings, foods, nutritive foods, functional foods and combinations thereof.

The compositions and combinations described herein can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments of any of the aspects, a film of lipid components is further hydrated with an aqueous solution of a drug to a form lipid layer encapsulating the drug, as described in U.S. Pat. No. 5,262,564.

For solid compositions, excipients include pharmaceutical grades of mannitol, lactose, starch, magnesium stearate, sodium saccharin, talcum, cellulose, glucose, sucrose, magnesium carbonate, and the like may be used. It is also possible for the compositions and combinations described herein to be coupled with soluble polymers as targetable drug carriers. Such polymers can include polyvinylpyrrolidone, pyran copolymer, polyhydroxypropyl-methacrylamide-phenol, polyhydroxyethylaspanamide-phenol, or polyethyleneoxidepolylysine substituted with palmitoyl residues. Furthermore, the compositions and combinations described herein may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin; (22) C₂-C₁₂ alcohols, such as ethanol; (23) silicone dioxide, and (24) other non-toxic compatible substances employed in pharmaceutical formulations. In some embodiments of any of the aspects, the excipient comprises, consists of, or consists essentially of silicone dioxide. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent.

In some embodiments of any of the aspects, the composition or combination described herein is provided or present as a single composition, e.g., each of the elements of the composition or combination are present in a single physical preparation, e.g., a single pill, tablet, gel, film, liquid, or solution.

In some embodiments of any of the aspects, the composition or combination described herein is provided or present as combination, set, or kit of multiple physical compositions, e.g., the elements of the composition or combination are present in a separate physical preparations, e.g., a separate pill, tablet, gel, film, liquid, or solution. The multiple physical compositions or preparations can be mixed prior to administration or consumption and/or administered or consumed as separate physical compositions or preparations.

In some embodiments of any of the aspects, the composition or combination does not comprise a live microbe. In some embodiments of any of the aspects, the composition or combination does not comprise an intact microbe, e.g., it does not comprise a killed microbe but can comprise portions or isolated/purified elements of a microbe. In some embodiments of any of the aspects, the composition or combination does not comprise a live yeast. In some embodiments of any of the aspects, the composition or combination does not comprise an intact yeast, e.g., it does not comprise a killed yeast but can comprise portions or isolated/purified elements of a yeast. In some embodiments of any of the aspects, the composition or combination does not comprise a live bacterium. In some embodiments of any of the aspects, the composition or combination does not comprise an intact bacterium, e.g., it does not comprise a killed microbe but can comprise portions or isolated/purified elements of a bacterium.

In some embodiments of any of the aspects, the composition or combination is sterilized, pasteurized, or thermized, e.g., irradiated, pascalized, UHT sterilized, pulsed electric field (PEF) pasteurized, microwave volumetric heating (MVH) pasteurized, or low temperature short time (LTST) pasteurized.

In some embodiments of any of the aspects, the composition or combination comprises a live microbe. In some embodiments of any of the aspects, the composition or combination comprises a live probiotic microbe. Non-limiting examples of probiotic microbes include Lactobacillus spp; Leuconostoc spp.; Bifidobacterium spp.; Lactococcus spp.; Lactobacillus bulgaricus; Leuconostoc mesenteroides; Lactobacillus plantarum; Pediococcus pentosaceus; Lactobacillus brevis; Leuconostoc citreum, Leuconostoc argentinum; Lactobacillus paraplantarum; Lactobacillus coryniformis; Weissella spp; L. pentosus; L. lactis; L. fermentum; Lactobacillus acidophilus, Bifidobacterium bifidum, Streptococcus thermophilus, Lactobacillus delbrueckii subsp. Bulgaricus; Lactobacillus helveticus; Lactobacillus kefiranofaciens, Lactobacillus rhamnosus; Lactobacillus casei; Lactobacillus johnsonii; Saccharomyces boulardii; Gluconacetobacter xylinus; Zygosaccharomyces sp.; Acetobacter pasteurianus; A. aceti; and Gluconobacter oxydans. In some embodiments of any of the aspects, the composition or combination comprises one or more live microbes selected from Aspergillus spp; Kluyveromyces spp; Aspergillus niger; Aspergillus oryzae; Kluyveromyces marxianus; Kluyveromyces marxianus CBS6014; Penicillium spp; Lactobacillus spp.; Aspergillus luchuensis; Saccharomyces spp; Aspergillus fumigatus; Saccharomyces cerevisiae; Kluyveromyces lactis; Streptomyces griseoflavus; Pseudomas spp.; Escherichia spp.; Leuconostoc spp; Gluconobacter spp.; S. cerevisiae, P. fluorescens, E. coli, L. mesenteroides, G. oxydans; Acidothermus spp.; Streptomyces sp. H7775; Streptomyces murinus; Acidothermus cellulolyticus; Thermobifida spp.; and Thermobifida fusca. In some embodiments of any of the aspects, the composition or combination comprises one or more live microbes selected from Penicillium spp; Bacillus spp.; P. fluorescens. A live microbe can be a wild-type or transgenic or genetically modified microbe. In some embodiments of any of the aspects, one or more enzymes of the composition or combination are produced, expressed by, or present in the one or more live microbes. In some embodiments of any of the aspects, the one or more live microbes do not produce, express, or comprise the one or more enzymes of the composition or combination.

In some embodiments of any of the aspects, the composition or combination described herein does not comprise flour, grains, or vegetable (e.g., plant) material. Flour and vegetable material are meant to refer to materials obtained from, respectively grains and plants, which is subject to chopping, grinding, or other gross processing methods and which comprise at least 30% of the different compounds present in the original material. Flour and vegetable material do not refer to isolated or purified materials obtained from plant origins, e.g., soluble starch.

In some embodiments of any of the aspects, the composition or combination further comprises fructose dehydrogenase, e.g., 5-D-fructose dehydrogenase.

In some embodiments of any of the aspects, the composition or combination does not comprise an invertase. In some embodiments of any of the aspects, the composition or combination does not comprise an exo-inulinase. In some embodiments of any of the aspects, the composition or combination does not comprise a beta-fructofuranosidase. In some embodiments of any of the aspects, the composition or combination does not comprise a beta-fructosidase.

In some embodiments of any of the aspects, the composition or combination does not comprise an alpha-galactosidase, a beta-glucanase, and/or a pectinase.

In one aspect of any of the embodiments, described herein is a kit comprising one or more of the compositions or combinations described herein. If the kit comprises multiple compositions and/or a combination of multiple elements (e.g., agents or enzymes), the multiple elements can be present in the same formulation of the kit or in separate formulations of the kit, e.g., for separate administration or for mixing prior to administration.

A kit is any manufacture (e.g., a package or container) comprising at least one agent, e.g., an enzyme, the manufacture being promoted, distributed, or sold as a unit for performing the methods described herein. The kits described herein can optionally comprise additional components useful for performing the methods described herein. By way of example, the kit can comprise measuring devices, or dosage guides, suitable for performing one or more of the administrations according to the methods described herein, an instructional material which describes performance of a method as described herein, and the like. Additionally, the kit may comprise an instruction leaflet.

In some embodiments of any of the aspects, where the combination or composition is formulated as a powder, the kit can comprise a dispenser for the powder, e.g., a shaker; a spoon sized or marked for a desired dosage; single dosage packets; resealable tubs, jars, or bags;

In a combination of elements (e.g., agents and/or enzymes), the different elements can be provided in a mixture or single formulation. Alternatively, the different elements can be provided in separate formulations that are packaged or provided as a set or kit.

In some embodiments of any of the aspects, the enzyme(s) present in a composition, or combination, of the disclosure exhibit an increased utility that is not exhibited when said enzyme(s) occur alone or when said enzyme(s) are present at a naturally occurring concentration. In some embodiments of any of the aspects, compositions of the disclosure, comprising two or more enzyme(s) as taught herein, exhibit a synergistic effect on imparting at least one improved digestive trait in a patient. In some embodiments of any of the aspects, the compositions of the disclosure comprising one or more enzyme(s) as taught herein exhibit markedly different characteristics/properties compared to their closest naturally occurring counterpart. That is, the compositions of the disclosure exhibit markedly different functional and/or structural characteristics/properties, as compared to their closest naturally occurring counterpart. For instance, the enzyme(s) of the disclosure are structurally different from an enzyme(s) as it naturally exists in a source organism (e.g., a bacterium, yeast, or mammal), for at least the following reasons: said enzyme(s) can be isolated and purified, such that it is not found in the milieu of the source organism, said enzyme(s) can be present at concentrations that do not occur in the source organism, said enzyme(s) can be associated with acceptable carriers that do not occur in the source organism, said enzyme(s) can be formulated to be shelf-stable and exist outside the source organism environment, and said enzyme(s) can be combined with other enzyme(s) at concentrations that do not exist in the source organism. Further, the enzyme(s) of the disclosure are functionally different from an enzyme(s) as it naturally exists in a source organism, for at least the following reasons: said enzyme(s) when applied in an isolated and purified form can lead to improved digestion, said enzyme(s) can be formulated to be shelf-stable and able to exist outside the source organism environment, such that the enzyme(s) now has a new utility as a supplement capable of administration to a patient, wherein the enzyme(s) could not have such a utility in its natural state in the source organism, as the enzyme(s) would be unable to survive outside the source organism without the intervention of the hand of man to formulate the enzyme(s) into a shelf-stable state and impart this new utility that has the aforementioned functional characteristics not possessed by the enzyme(s) in its natural state of existence in the source organism.

In one aspect of any of the embodiments, provided herein is a nucleic acid comprising a sequence encoding one or more of: at least one fructan-degrading enzyme; at least one galactosidase; at least one sorbitol-degrading enzyme; at least one mannitol-degrading enzyme; at least one glucose isomerase; and at least one mannose isomerase; and at least one of: an operably linked heterologous promoter; and at least one codon optimized for expression in bacteria and/or yeast. In one aspect of any of the embodiments, provided herein is a nucleic acid comprising a sequence encoding one or more of: at least one fructan-degrading enzyme; at least one galactosidase; at least one sorbitol-degrading enzyme; and at least one mannitol-degrading enzyme; and at least one of: an operably linked heterologous promoter; and at least one codon optimized for expression in bacteria and/or yeast.

In one aspect of any of the embodiments, provided herein is a cell (e.g., a bacterial or yeast cell) comprising a nucleic acid comprising a sequence encoding one or more of: at least one fructan-degrading enzyme; at least one galactosidase; at least one sorbitol-degrading enzyme; at least one mannitol-degrading enzyme; at least one glucose isomerase; and at least one mannose isomerase; and at least one of: an operably linked heterologous promoter; and at least one codon optimized for expression in bacteria and/or yeast. In one aspect of any of the embodiments, provided herein is a cell (e.g., a bacterial or yeast cell) comprising nucleic acid comprising a sequence encoding one or more of: at least one fructan-degrading enzyme; at least one galactosidase; at least one sorbitol-degrading enzyme; and at least one mannitol-degrading enzyme; and at least one of: an operably linked heterologous promoter; and at least one codon optimized for expression in bacteria and/or yeast.

In some embodiments of any of the aspects, the nucleic acid is provided in a vector, e.g., an expression vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence that is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid described herein, or portion thereof as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo or in the transduced cells. It should be understood that the vectors described herein can, in some embodiments, be combined with other suitable compositions and therapies. In some embodiments of any of the aspects, the vector is episomal. The use of a suitable episomal vector provides a means of maintaining the nucleotide of interest in the subject in high copy number extra chromosomal DNA thereby eliminating potential effects of chromosomal integration.

In one aspect of any of the embodiments, described herein is a composition comprising an effective amount of an enzyme mixture including a subset or all of, but not limited to the following enzymes:
a) exo-inulinase obtained from a source such as but not limited to Aspergillus oryzae and Kluyveromyces marxianus
b) endo-inulinase obtained from a source such as but not limited to Aspergillus niger
c) mannitol dehydrogenase obtained from a source such as but not limited to Pseudomonas fluorescens, E. coli, Leuconostoc mesenteroides, and Gluconobacter oxydans, where “mannitol dehydrogenase” refers to any enzyme whose catalytic activity includes D-mannitol as substrate and D-fructose or D-mannose as product, and may include but is not limited to mannitol dehydrogenase, mannitol 2-dehydrogenase and mannitol dehydrogenase 2
d) sorbitol dehydrogenase obtained from a source such as but not limited to rat liver and sheep liver, where “sorbitol dehydrogenase” refers to any enzyme whose catalytic activity includes D-sorbitol as substrate and D-fructose as product and may include but is not limited to sorbitol dehydrogenase, sorbitol dehydrogenase 1, and sorbitol dehydrogenase 2 Embodiments which contain (a) and/or (b) of the list above are intended to hydrolyze fructan molecules found in foods and beverages during the consumption and digestion of such foods into more readily absorbable components (glucose and fructose). Embodiments which contain (c) of the list above are intended to convert mannitol into absorbable molecules (fructose and/or mannose). Embodiments which contain (d) of the list above are intended to convert sorbitol into absorbable fructose molecules. Embodiments of the present invention may hold more than one purpose, depending on the composition of enzymes appearing in the embodiment.

In some embodiments of any of the aspects, the composition described herein can contain auxiliary agents to support activity of the enzymes used in the composition including but not limited to metal ions (such as but not limited to Fe+3, Cu+3, and Mn+2), antacids, H2 antagonists, proton pump inhibitors, pepstatin protease inhibitors, other artificial protease inhibitors, and protease inhibitors naturally occurring in and extracted from foods such as but not limited to lima beans, soybeans, and chickpeas. Metal ions are demonstrated in scientific literature to potentially elevate enzymatic activity. Antacids can reduce symptoms of indigestion in users such as heartburn or dyspepsia. Antacids, H2 antagonists, and proton pump inhibitors can also increase the pH of the stomach, enabling the enzymes to activate closer to their optimal pH range (usually in the 4-8 range). Protease inhibitors may decrease the activity of endogenous proteases within the human gastrointestinal tract such as pepsin and trypsin, and prevent their cleavage and deactivation of the enzymes in the composition pertaining to the present invention. Additionally, in the scientific literature, it has been suggested that protease inhibition may lend therapeutic effects to individuals suffering from gastrointestinal conditions, including IBS, IBD, and SIBO. The composition may also contain additional enzymes not intended to act on fructan, mannitol, or sorbitol. These additional enzymes include but are not limited to glucose isomerase and mannose isomerase. In particular, glucose isomerase will convert excess fructose molecules produced by the reactions catalyzed by the enzymes in the embodiment's composition into glucose molecules, which are digested more quickly by the body than fructose molecules, and as a result, shift the equilibrium of the aforementioned reactions to facilitate greater enzymatic activity. Similarly, mannose isomerase will convert excess mannose produced by the reactions catalyzed by mannitol dehydrogenase into fructose, which may consequently be converted into glucose by glucose isomerase present in the composition. Ultimately, the objective is to remove excess mannose via a series of conversions that transform the molecule into one that is quickly absorbed (glucose), to encourage higher enzymatic activity.

An embodiment may also contain a non-toxic and safe-for-consumption excipient, and may be produced in tablet (including chewable), capsule, soft-gel, thin-film, powder, or liquid or syrup form, but is not limited to such embodiments. The form that contains the composition may be in its entirety enteric-coated to reserve enzymatic activity for the intestinal tract, or contain a portion that is enteric-coated, so as to activate before reaching the small intestine but also release enzymes for continued activity in the intestinal tract. The composition may be mixed, blended, or mashed into food before ingestion, or be ingested in any of, but not limited to, the above forms in one or multiple doses or instances of administration within a period of time beginning 30 minutes prior to a meal and 30 minutes after a meal.

In some embodiments of any of the aspects, the composition is provided as a food, drink, or other nutritional product, e.g., a prepared or processed food or drink. It is contemplated that inclusion of the one or more enzymes described herein in the food, drink, or other product will provide dosing of a subject at the same time that the FODMAP is consumed, and/or reduce the FODMAP content of the food, drink, or other product. Exemplary but non-limiting examples of foods, drinks, and other nutritional products include prepared foods including microwavable, ready-to-heat, ready-to-bake, or ready-to-serve soups, condiments, entrées, sides, salads, snacks, desserts (which may be canned, bottled, frozen, fresh, or in any format or packaging, but is not limited to packaged products); juice, jam, fruit-based beverages, teas, fruit sauces (including but not limited to apple sauce), canned fruit and vegetables, fermented fruits and vegetables (including but not limited to kimchi, sauerkraut), fruit and vegetable snacks (including but not limited to fruit leather, gummies, vegetable chips/fries/straws), smoothies, confectionaries (including but not limited to candy chews, marzipan, candy bars), dips and spread (including but not limited to hummus, guacamole), sauces (including but not limited to mayonnaise ketchup, salad dressings, mustard), marinades, nut butters, fruit or vegetable purées or pastes, nut milks, snack bars, bouillons, broth, soup base, seasonings, drink powders, beer, wine, liquor, prepared meats (including but not limited to pâté, sausages, salami), breads, crackers, pastries (including but not limited to croissants, muffins, bagels), baking flours, baking mixes, any form of frozen dessert (including but not limited to popsicles, sorbets, ice cream); meal kits intended for preparation at home; and quick-cook packaged products (including but not limited to instant noodles, instant mac and cheese).

In one aspect of any of the embodiments, described herein is a method (e.g., a non-therapeutic method) of improving digestion, the method comprising consuming, or administering to a subject, the composition or combination described herein. In some embodiments of any of the aspects, a method of improving digestion is not a therapeutic method nor a method of medical treatment, as a method of improving digestion does not require that the subject have a pathological condition which is treated. In some embodiments of any of the non-therapeutic aspects, the subject is a healthy subject. In some embodiments of any of the non-therapeutic aspects, the subject does not have and/or is not diagnosed as having a medical condition affecting digestion.

In one aspect of any of the embodiments, described herein is a method of treating a digestive condition in a subject in need thereof, the method comprising administering to a subject, the composition or combination described herein. In one aspect of any of the embodiments, provided herein is the composition or combination described herein for use in a method of treating a digestive condition in a subject. In some embodiments of any of the aspects, administering can comprise directing the subject to consume the composition and/or combination.

As used herein, “digestive condition” refers to a condition in which digestion of one or more foods is impaired and/or causes symptoms of bloating, gas, nausea, abdominal pain, abdominal discomfort, diarrhea, and/or constipation. Exemplary but non-limiting digestive conditions can include Irritable Bowel Syndrome (IBS), Inflammatory Bowel Disease (IBD), non-celiac gluten sensitivity, small intestinal bacterial overgrowth (SIBO), small intestinal fungal overgrowth (SIFO) and/or allium intolerance. In some embodiments of any of the aspects, the subject is or is diagnosed as being sensitive to one or more FODMAPs.

Crohn's disease and ulcerative colitis affect different regions of the intestines (also referred to herein as the bowels). For example, Crohn's disease affects both the small intestine and the large intestine (also referred to herein as the colon); in Crohn's disease, affected portions of the intestine are intermixed with healthy portions of the intestine; and Crohn's disease can occur in all three layers of the bowel walls. As another example, ulcerative colitis affects only the colon; ulcerative colitis involves continuous inflammation of a majority of or the entire colon; and ulcerative colitis only affects the inner most lining of the colon.

Ulcerative colitis and Crohn's disease can exhibit similar symptoms. For example, symptoms of ulcerative colitis or Crohn's disease can comprise abdominal pain, diarrhea (sometimes bloody and/or containing mucus), bloating, abdominal cramps, urgency to have a bowel movement (defecate), anorexia, nausea/vomiting, constipation, and/or weight loss. As ulcerative colitis and Crohn's disease can exhibit similar symptoms, doctors frequently cannot distinguish either disease by symptoms alone.

In some embodiments of any of the aspects, the method provides a decrease in bloating, gas, abdominal pain, abdominal discomfort, nausea, diarrhea, and/or constipation. In some embodiments of any of the aspects, the dose is a dose effective in providing a decrease in bloating, gas, nausea, abdominal pain, abdominal discomfort, diarrhea, and/or constipation.

In some embodiments of any of the aspects, the methods described herein relate to treating a subject having or diagnosed as having a digestive condition with a composition or combination as described herein. Subjects having a digestive condition can be identified by a physician using current methods of diagnosing a digestive condition.

The compositions and methods described herein can be administered to a subject having or diagnosed as having a digestive condition. In some embodiments of any of the aspects, the methods described herein comprise administering an effective amount of compositions described herein, e.g. a composition or combination, to a subject in order to alleviate a symptom of a digestive condition. As used herein, “alleviating a symptom” is ameliorating any condition or symptom associated with the digestive condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art.

The term “effective amount” as used herein refers to the amount of a composition or combination described herein needed to alleviate at least one or more symptom of the disease or disorder, and relates to a sufficient amount of pharmacological composition to provide the desired effect. The term “therapeutically effective amount” therefore refers to an amount of the active ingredient(s) that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient(s), which achieves a half-maximal inhibition of symptoms) as determined in cell culture, or in an appropriate animal model. Levels in plasma can be measured, for example, by high performance liquid chromatography. The effects of any particular dosage can be monitored by a suitable assay or monitoring of symptoms. The dosage can be determined by a physician and adjusted, as necessary, to suit observed effects of the treatment.

In some embodiments of any of the aspects, the composition or combination described herein is administered as a monotherapy, e.g., another treatment for the digestive condition is not administered to the subject. In some embodiments of any of the aspects, the methods described herein can further comprise administering a second agent and/or treatment to the subject, e.g. as part of a combinatorial therapy.

In certain embodiments, an effective dose of a composition or combination as described herein can be administered to a patient once. In certain embodiments, an effective dose of a composition of a combination described herein can be administered to a patient repeatedly.

In some embodiments of any of the aspects, subjects can be administered a therapeutic amount of a composition or combination described herein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments of any of the aspects, subjects can be administered a therapeutic amount of a composition or combination described herein, e.g., between 1 and 10,000,000 units of enzymatic activity for each enzyme per dose. In some embodiments of any of the aspects, the therapeutic amount of a composition or combination described herein comprises between 1 and 2,000,000 units of enzymatic activity for each enzyme per dose. In some embodiments of any of the aspects, the therapeutic amount of a composition or combination described herein comprises between 1 and 1,000,000 units of enzymatic activity for each enzyme per dose. In some embodiments of any of the aspects, the therapeutic amount of a composition or combination described herein comprises between 1 and 500,000 units of enzymatic activity for each enzyme per dose. In some embodiments of any of the aspects. In some embodiments of any of the aspects, the therapeutic amount of a composition or combination described herein comprises between 1 and 200,000 units of enzymatic activity for each enzyme per dose. In some embodiments of any of the aspects, In some embodiments of any of the aspects, the therapeutic amount of a composition or combination described herein comprises between 1 and 100,000 units of enzymatic activity for each enzyme per dose. In some embodiments of any of the aspects, the therapeutic amount of a composition or combination described herein comprises between 500 and 1,000,000 units of enzymatic activity for each enzyme per dose. In some embodiments of any of the aspects, the therapeutic amount of a composition or combination described herein comprises between 500 and 500,000 units of enzymatic activity for each enzyme per dose. In some embodiments of any of the aspects,

Units of enzymatic activity can be defined as:
a. One Unit of exo-inulinase activity is defined as the amount of enzyme required to release one μmole of β-D-fructose reducing-sugar equivalents per minute from kestose (5 mg/mL) in sodium acetate buffer (100 mM), at pH 4.5 at 40 °C
b. One Unit of endo-inulinase activity is defined as the amount of enzyme required to release one μmole of β-D-fructose reducing-sugar equivalents per minute from inulin (20 mg/mL) in sodium acetate buffer (100 mM), pH 4.5.
c. Glucose isomerase: one unit converts glucose to fructose at an initial rate of 1 μmole per min
d. One Unit of mannitol dehydrogenase is defined as the amount of enzyme required to produce one μmole of NADH from NAD+ in the presence of D-mannitol (5.2 mM) in Tris-HCl buffer (64 mM), pH 8.6 at 40 °C
e. One unit (U) sorbitol dehydrogenase will reduce 1 mol of D-fructose in one minute at +25 °C and pH 7.6 [triethanolamine buffer; 150 mM fructose (nonsaturating concentration)].
f. Unit definition of Mannitol Oxidase=One unit will oxidize 1.0 μmole of D-mannitol to D-mannose and H₂O₂ per minute at pH 5.1 at 35 °C
g. Unit definition of Sorbitol Oxidase=One unit will oxidize 1.0 μmole of D-sorbitol to D-glucose and H₂O₂ per minute at pH 5.1 at 35 °C.

In some embodiments of any of the aspects, the therapeutic amount of a composition or combination described herein comprises between 10 and 100,000 units of glucose isomerase activity per dose. In some embodiments of any of the aspects, the therapeutic amount of a composition or combination described herein comprises between 100 and 10,000 units of glucose isomerase activity per dose. In some embodiments of any of the aspects, the therapeutic amount of a composition or combination described herein comprises between 500 and 5,000 units of glucose isomerase activity per dose.

In some embodiments of any of the aspects, the therapeutic amount of a composition or combination described herein comprises between 50 and 500,000 units of galactosidase activity per dose. In some embodiments of any of the aspects, the therapeutic amount of a composition or combination described herein comprises between 500 and 50,000 units of galactosidase activity per dose. In some embodiments of any of the aspects, the therapeutic amount of a composition or combination described herein comprises between 1,000 and 10,000 units of galactosidase activity per dose. In some embodiments of any of the aspects, the therapeutic amount of a composition or combination described herein comprises between 2,000 and 10,000 units of galactosidase activity per dose. In some embodiments of any of the aspects, the therapeutic amount of a composition or combination described herein comprises between 3,000 and 10,000 units of galactosidase activity per dose. In some embodiments of any of the aspects, the therapeutic amount of a composition or combination described herein comprises between 4,000 and 10,000 units of galactosidase activity per dose. In some embodiments of any of the aspects, the therapeutic amount of a composition or combination described herein comprises between 5,000 and 10,000 units of galactosidase activity per dose. In some embodiments of any of the aspects, the therapeutic amount of a composition or combination described herein comprises at least 2,000 units of galactosidase activity per dose. In some embodiments of any of the aspects, the therapeutic amount of a composition or combination described herein comprises at least 3,000 units of galactosidase activity per dose. In some embodiments of any of the aspects, the therapeutic amount of a composition or combination described herein comprises at least 4,000 units of galactosidase activity per dose. In some embodiments of any of the aspects, the therapeutic amount of a composition or combination described herein comprises at least 5,000 units of galactosidase activity per dose.

In some embodiments of any of the aspects, the therapeutic amount of a composition or combination described herein comprises between 800 and 8,000,000 units of fructan-degrading enzyme activity (e.g., exo-inulinase activity) per dose. In some embodiments of any of the aspects, the therapeutic amount of a composition or combination described herein comprises between 8,000 and 800,000 units of fructan-degrading enzyme activity (e.g., exo-inulinase activity) per dose. In some embodiments of any of the aspects, the therapeutic amount of a composition or combination described herein comprises between 4,000 and 400,000 units of fructan-degrading enzyme activity (e.g., exo-inulinase activity) per dose. In some embodiments of any of the aspects, the therapeutic amount of a composition or combination described herein comprises between 1,000 and 10,000 units of fructan-degrading enzyme activity (e.g., exo-inulinase activity) per dose. In some embodiments of any of the aspects, the therapeutic amount of a composition or combination described herein comprises between 1,000 and 20,000 units of fructan-degrading enzyme activity (e.g., exo-inulinase activity) per dose.

In some embodiments of any of the aspects, the therapeutic amount of a composition or combination described herein comprises between 100 and 500,000 units of lactase activity per dose. In some embodiments of any of the aspects, the therapeutic amount of a composition or combination described herein comprises between 1,000 and 100,000 units of lactase activity per dose. In some embodiments of any of the aspects, the therapeutic amount of a composition or combination described herein comprises between 10,000 and 50,000 units of lactase activity per dose. In some embodiments of any of the aspects, the therapeutic amount of a composition or combination described herein comprises between 15,000 and 50,000 units of lactase activity per dose. In some embodiments of any of the aspects, the therapeutic amount of a composition or combination described herein comprises at least 10,000 units of lactase activity per dose. In some embodiments of any of the aspects, the therapeutic amount of a composition or combination described herein comprises at least 12,000 units of lactase activity per dose. In some embodiments of any of the aspects, the therapeutic amount of a composition or combination described herein comprises at least 15,000 units of lactase activity per dose. In some embodiments of any of the aspects, the therapeutic amount of a composition or combination described herein comprises at least 20,000 units of lactase activity per dose.

In some embodiments of any of the aspects, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. bloating, gas, constipation, etc. by at least 10%, at least 15%, at least 20%, at least 25%, at least 30%, at least 40%, at least 50%, at least 60%, at least 70%, at least 80% or at least 90% or more.

In some embodiments of any of the aspects, consumption or administration occurs within 30 minutes of consumption of a meal, or consumption of other food or drink. In some embodiments of any of the aspects, consumption or administration occurs within 30 minutes (e.g., within 20 minutes or within 10 minutes) prior to consumption of a meal, or consumption of other food or drink. In some embodiments of any of the aspects, consumption or administration occurs at least partially during consumption of a meal, or consumption of other food or drink.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient(s). The desired dose or amount of activation can be administered at one time or divided into sub doses, e.g., 2-4 sub doses and administered over a period of time, e.g., at appropriate intervals through the day or other appropriate schedule. In some embodiments of any of the aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition or combination can be administered over a period of time, such as over a 5 minute, 10 minute, 15 minute, 20 minute, or 25 minute period.

The dosage ranges for the administration of composition and combinations according to the methods described herein depend upon, for example, the form of the active ingredient(s), its potency, and the extent to which symptoms, markers, or indicators of a condition described herein are desired to be reduced, for example the percentage reduction desired for bloating, gas, constipation, etc. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition or combination in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of a mouse model of a digestive condition. When using an experimental animal model, efficacy of treatment is evidenced when a statistically significant change in a marker is observed, e.g. gas, bloating, constipation, or percent of one or more compounds digested.

The polypeptides (e.g., enzymes) of the present invention can be synthesized by using well known methods including recombinant methods and chemical synthesis. Recombinant methods of producing a polypeptide through the introduction of a vector including nucleic acid encoding the polypeptide into a suitable host cell are well known in the art, e.g., as described in Sambrook et al., Molecular Cloning: A Laboratory Manual, 2d Ed, Vols 1 to 8, Cold Spring Harbor, NY (1989); M. W. Pennington and B. M. Dunn, Methods in Molecular Biology: Peptide Synthesis Protocols, Vol 35, Humana Press, Totawa, NJ (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, NY (1984); Kimmerlin, T. and Seebach, D. J. Pept. Res. 65:229-260 (2005); Nilsson et al., Annu. Rev. Biophys. Biomol. Struct. (2005) 34:91-118; W. C. Chan and P. D. White (Eds.) Fmoc Solid Phase Peptide Synthesis: A Practical Approach, Oxford University Press, Cary, NC (2000); N. L. Benoiton, Chemistry of Peptide Synthesis, CRC Press, Boca Raton, FL (2005); J. Jones, Amino Acid and Peptide Synthesis, 2^{nd} Ed, Oxford University Press, Cary, NC (2002); and P. Lloyd-Williams, F. Albericio, and E. Giralt, Chemical Approaches to the synthesis of peptides and proteins, CRC Press, Boca Raton, FL (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

In some embodiments of any of the aspects, a polypeptide “obtained from” a specified organism refers to a polypeptide (e.g., an enzyme) isolated by physical or chemical means from that organism, e.g., purified in polypeptide from one or more cells of that organism. In some embodiments of any of the aspects, a polypeptide having the sequence of a reference polypeptide of a specified organism(s) refers to a polypeptide (e.g., an enzyme) produced or obtained by any means or source that comprises the same polypeptide sequence as a reference polypeptide produced by the specified organism, e.g., the polypeptide could be recombinantly produced.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments of any of the aspects, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of digestion and/or a digestive condition. A subject can be male or female.

A subject can be one who has been previously diagnosed with or identified as suffering from or having a condition in need of treatment (e.g. a digestive condition) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect, e.g. of the wild-type polypeptide, e.g., as described herein. Conservative substitution variants that maintain the activity of wildtype enzymes will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of digestion and/or a digestive condition as described herein.

In some embodiments of any of the aspects, a polypeptide (e.g. an enzyme), can be a variant of a sequence described herein. In some embodiments of any of the aspects, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of the wildtype enzyme, e.g. 110%, 125%, 150%, 175%, 200%, 500%, 1000% or more.

One method of identifying amino acid residues which can be substituted is to align, for example, a reference sequence to a homolog from one or more different species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp or BLASTn (available on the world wide web at blast.ncbi.nlm.nih.gov), with default parameters set.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity and specificity of a native or reference polypeptide is retained.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Typically conservative substitutions for one another also include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

In some embodiments of any of the aspects, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments of any of the aspects, the polypeptide described herein can be a variant of a sequence described herein. In some embodiments of any of the aspects, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments of any of the aspects, a polypeptide (e.g. enzyme) can comprise one or more amino acid substitutions or modifications. In some embodiments of any of the aspects, the substitutions and/or modifications can prevent or reduce proteolytic degradation and/or prolong half-life of the polypeptide in a subject. In some embodiments of any of the aspects, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments of any of the aspects, a polypeptide, e.g., an enzyme, as described herein can comprise at least one peptide bond replacement. A polypeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments of any of the aspects, a polypeptide, e.g., an enzyme, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H). In some embodiments of any of the aspects, a polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxy glutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments of any of the aspects, a polypeptide, e.g. an enzyme, can be modified, e.g. by addition of a moiety to one or more of the amino acids that together comprise the peptide. In some embodiments of any of the aspects, a polypeptide as described herein can comprise one or more moiety molecules, e.g. 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments of any of the aspects, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments of any of the aspects, an end-capping modification can comprise acetylation at the N-terminus, N-terminal acylation, and N-terminal formylation. In some embodiments of any of the aspects, an end-capping modification can comprise amidation at the C-terminus, introduction of C-terminal alcohol, aldehyde, ester, and thioester moieties. The half-life of a polypeptide can be increased by the addition of moieties, e.g. PEG, albumin, or other fusion partners (e.g. Fc fragment of an immunoglobin).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established. Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments of any of the aspects, a polypeptide as described herein can be chemically synthesized and mutations can be incorporated as part of the chemical synthesis process.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, an enzyme described herein is exogenous. In some embodiments of any of the aspects, an enzyme described herein is ectopic. In some embodiments of any of the aspects, an enzyme described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments of any of the aspects, a nucleic acid encoding a polypeptide as described herein (e.g. an enzyme) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments of any of the aspects, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a digestive condition. The term “treating” includes reducing or alleviating at least one adverse effect or symptom of a condition, disease or disorder associated with a digestive condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to symptoms induced by eating particular foodstuffs (e.g., FODMAPs). For example, the composition or combination can be consumed prior to eating/drinking other substances in order to prevent or reduce the severity of bloating, gas, constipation, etc. as described herein. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments of any of the aspects, administration comprises physical human activity, e.g., an injection, act of ingestion, an act of application, and/or manipulation of a delivery device or machine. Such activity can be performed, e.g., by a medical professional and/or the subject being treated.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A composition or combination comprising at least one of:
   a. At least one fructan-degrading enzyme;
   b. At least one galactosidase;
   c. At least one sorbitol-degrading enzyme;
   d. At least one mannitol-degrading enzyme;
   e. At least one glucose isomerase; and
   f. At least one mannose isomerase.
2. A composition or combination comprising at least one of:
   a. At least one fructan-degrading enzyme;
   b. At least one galactosidase;
   c. At least one sorbitol-degrading enzyme;
   d. At least one mannitol-degrading enzyme; and
   e. At least one glucose isomerase.
3. The composition or combination of numbered paragraph 1, comprising at least one of:
   a. At least one fructan-degrading enzyme;
   b. At least one galactosidase;
   c. At least one sorbitol-degrading enzyme; and
   d. At least one mannitol-degrading enzyme.
4. The composition or combination of numbered paragraph 1, comprising at least two of:
   a. At least one fructan-degrading enzyme;
   b. At least one galactosidase;
   c. At least one sorbitol-degrading enzyme; and
   d. At least one mannitol-degrading enzyme.
5. The composition or combination of numbered paragraph 1, comprising at least three of:
   a. At least one fructan-degrading enzyme;
   b. At least one galactosidase;
   c. At least one sorbitol-degrading enzyme; and
   d. At least one mannitol-degrading enzyme.
6. The composition or combination of numbered paragraph 1, comprising:
   a. At least one fructan-degrading enzyme;
   b. At least one galactosidase;
   c. At least one sorbitol-degrading enzyme; and
   d. At least one mannitol-degrading enzyme.
7. A composition or combination comprising:
   a. At least one fructan-degrading enzyme; and
   b. At least one galactosidase.
8. A composition or combination comprising:
   a. At least one fructan-degrading enzyme;
   b. At least one sorbitol-degrading enzyme;
   c. At least one mannitol-degrading enzyme; and
   d. At least one glucose isomerase.
9. A composition or combination comprising:
   a. At least one fructan-degrading enzyme;
   b. At least one galactosidase; and
   c. At least one glucose isomerase.
10. A composition or combination comprising:
   a. At least one fructan-degrading enzyme;
   b. At least one alpha-galactosidase; and
   c. At least one lactase.
11. A composition or combination comprising:
   a. At least one fructan-degrading enzyme;
   b. At least one galactosidase;
   c. At least one sorbitol-degrading enzyme;
   d. At least one mannitol-degrading enzyme;
   e. At least one glucose isomerase; and
   f. At least one mannose isomerase.
12. The composition or combination of any of the preceding numbered paragraphs, wherein the at least one fructan-degrading enzyme comprises an inulinase, and optionally, has a sequence with at least 80% sequence identity to one of SEQ ID NOs: 305-799.
13. The composition or combination of numbered paragraph 12, wherein the inulinase is a yeast inulinase.
14. The composition or combination of numbered paragraph 12, wherein the inulinase is an exo-inulinase.
15. The composition or combination of numbered paragraph 14, wherein the exo-inulinase is an exo-inulinase of, or is obtained from an Aspergillus or Kluyveromyces spp.
16. The composition or combination of numbered paragraph 14, wherein the exo-inulinase is an exo-inulinase of, or is obtained from Aspergillus oryzae or Kluyveromyces marxianus.
17. The composition or combination of numbered paragraph 14, wherein the exo-inulinase is an exo-inulinase of, or is obtained from Kluyveromyces marxianus CBS6014.
18. The composition or combination of numbered paragraph 14, wherein the exo-inulinase has a sequence with at least 80% sequence identity to one of SEQ ID NOs: 3-7, 29-31, and 42-45.
19. The composition or combination of numbered paragraph 12, wherein the inulinase is an endo-inulinase.
20. The composition or combination of numbered paragraph 19, wherein the endo-inulinase is an endo-inulinase of, or is obtained from an Aspergillus spp.
21. The composition or combination of numbered paragraph 20, wherein the endo-inulinase is an endo-inulinase of, or is obtained from Aspergillus niger.
22. The composition or combination of numbered paragraph 21, wherein the endo-inulinase has a sequence with at least 80% sequence identity to one of SEQ ID NOs: 1-2.
23. The composition or combination of any of the preceding numbered paragraphs, comprising both an exo-inulinase and an endo-inulinase.
24. The composition or combination of any of the preceding numbered paragraphs, wherein the at least one fructan-degrading enzyme comprises a levanase.
25. The composition or combination of numbered paragraph 24, wherein the inulinase is a bacterial levanase.
26. The composition or combination of numbered paragraph 25, wherein the inulinase is an exo-levanase.
27. The composition or combination of numbered paragraph 26, wherein the exo-levanase is an exo-levanase of, or is obtained from an Gluconacetobacter spp.
28. The composition or combination of numbered paragraph 26, wherein the exo-levanase is an exo-levanase of, or is obtained from Gluconacetobacter johannae.
29. The composition or combination of numbered paragraph 26, wherein the exo-levanase has a sequence with at least 80% sequence identity to SEQ ID NO: 50.
30. The composition or combination of numbered paragraph 24, wherein the levanase is an endo-levanase.
31. The composition or combination of numbered paragraph 30, wherein the endo-levanase is an endo-levanase of, or is obtained from an Bacillus spp.
32. The composition or combination of numbered paragraph 30, wherein the endo-levanase is an endo-levanase of, or is obtained from Bacillus spizizenii.
33. The composition or combination of numbered paragraph 30, wherein the endo-levanase has a sequence with at least 80% sequence identity to SEQ ID NO 49.
34. The composition or combination of any of the preceding numbered paragraphs, comprising both an exo-levanase and an endo-levanase.
35. The composition or combination of any of the preceding numbered paragraphs, wherein the at least one fructan-degrading enzyme comprises an amyloglucosidase.
36. The composition or combination of numbered paragraph 35, wherein the amyloglucosidase comprises a sequence with at least 80% sequence identity to one of SEQ ID NOs: 8-10.
37. The composition or combination of any of the preceding numbered paragraphs, wherein the at least one galactosidase comprises an alpha-galactosidase.
38. The composition or combination of numbered paragraph 37, wherein the alpha-galactosidase is an alpha-galactosidase of, or is obtained from an Aspergillus spp.
39. The composition or combination of numbered paragraph 37, wherein the alpha-galactosidase is an alpha-galactosidase of, or is obtained from Aspergillus niger.
40. The composition or combination of numbered paragraph 37, wherein the alpha-galactosidase comprises a sequence with at least 80% sequence identity to one of SEQ ID NOs: 11-14.
41. The composition or combination of any of the preceding numbered paragraphs, wherein the at least one galactosidase comprises a beta-galactosidase.
42. The composition or combination of numbered paragraph 41, wherein the beta-galactosidase is a beta-galactosidase of, or is obtained from an Aspergillus spp.
43. The composition or combination of numbered paragraph 41, wherein the beta-galactosidase is a beta-galactosidase of, or is obtained from Aspergillus niger.
44. The composition or combination of numbered paragraph 41, wherein the beta-galactosidase comprises a sequence with at least 80% sequence identity to SEQ ID NO: 15.
45. The composition or combination of any of the preceding numbered paragraphs, wherein the at least one galactosidase comprises a lactase.
46. The composition or combination of numbered paragraph 10 or 45, wherein the lactase is a lactase of, or is obtained from an Kluyveromyces or Aspergillus spp.
47. The composition or combination of numbered paragraph 10 or 45, wherein the lactase is a lactase of, or is obtained from Kluyveromyces lactis, Kluyveromyces marxianus, Aspergillus niger, or Aspergillus oryzae.
48. The composition or combination of numbered paragraph 10 or 45, wherein the lactase comprises a sequence with at least 80% sequence identity to one of SEQ ID NOs: 15 or 36-41.
49. The composition or combination of any of the preceding numbered paragraphs, wherein the at least one galactosidase comprises an alpha-galactosidase and a beta-galactosidase.
50. The composition or combination of any of the preceding numbered paragraphs, wherein the at least one sorbitol-degrading enzyme comprises a sorbitol dehydrogenase.
51. The composition or combination of numbered paragraph 50, wherein the sorbitol dehydrogenase is a mammalian sorbitol dehydrogenase.
52. The composition or combination of numbered paragraph 50, wherein the sorbitol dehydrogenase is a sorbitol dehydrogenase of, or is obtained from a Ovis or Rattus species.
53. The composition or combination of numbered paragraph 50, wherein the sorbitol dehydrogenase has a sequence with at least 80% sequence identity to one of SEQ ID NOs: 16-18.
54. The composition or combination of any of the preceding numbered paragraphs, wherein the at least one sorbitol-degrading enzyme comprises a sorbitol oxidase.
55. The composition or combination of numbered paragraph 54, wherein the sorbitol oxidase is a yeast sorbitol oxidase.
56. The composition or combination of numbered paragraph 54, wherein the sorbitol oxidase has a sequence with at least 80% sequence identity to at least one of SEQ ID NOs: 19, 32-35, and 51-304.
57. The composition or combination of any of the preceding numbered paragraphs, wherein the at least one mannitol-degrading enzyme comprises a mannitol dehydrogenase.
58. The composition or combination of numbered paragraph 57, wherein the mannitol dehydrogenase is a bacterial mannitol dehydrogenase.
59. The composition or combination of numbered paragraph 58, wherein the mannitol dehydrogenase is a mannitol dehydrogenase of, or is obtained from a Pseudomonas, Escherichia, Leuconostoc, or Gluconobacter spp.
60. The composition or combination of numbered paragraph 58, wherein the mannitol dehydrogenase is a mannitol dehydrogenase of, or is obtained from P. fluorescens, E. coli, L. mesenteroides, or G. oxydans.
61. The composition or combination of numbered paragraph 58, wherein the mannitol dehydrogenase has a sequence with at least 80% sequence identity to one of SEQ ID NOs: 20-26.
62. The composition or combination of numbered paragraph 57, wherein the at least one mannitol-degrading enzyme comprises a mannitol oxidase.
63. The composition or combination of numbered paragraph 62, wherein the mannitol oxidase has a sequence with at least 80% sequence identity to at least one of SEQ ID NOs: 32-35 and 51-304.
64. The composition or combination of any of the preceding numbered paragraphs, wherein the glucose isomerase is a yeast glucose isomerase.
65. The composition or combination of numbered paragraph 64, wherein the glucose isomerase is a glucose isomerase of, or is obtained from a Streptomyces spp.
66. The composition or combination of numbered paragraph 64, wherein the glucose isomerase is a glucose isomerase of, or is obtained from S. murinus.
67. The composition or combination of numbered paragraph 64, wherein the glucose isomerase has a sequence with at least 80% sequence identity to SEQ ID NO: 27.
68. The composition or combination of any of the preceding numbered paragraphs, wherein the mannose isomerase is a bacterial mannose isomerase.
69. The composition or combination of numbered paragraph 68, wherein the mannose isomerase is a mannose isomerase of, or is obtained from a Thermobifida spp.
70. The composition or combination of numbered paragraph 68, wherein the mannose isomerase is a mannose isomerase of, or is obtained from T. fusca.
71. The composition or combination of numbered paragraph 68, wherein the mannose isomerase has a sequence with at least 80% sequence identity to SEQ ID NO: 28.
72. The composition or combination of any of the preceding numbered paragraphs, further comprising one or more of: a cofactor; a metal ion; an antacid; an H₂ antagonist; a proton pump inhibitor; a pepstatin protease inhibitor; or a protease inhibitor.
73. The composition or combination of any of the preceding numbered paragraphs, formulated as a food supplement.
74. The composition or combination of any of the preceding numbered paragraphs, packaged as or as part of a food, drink, or other nutritional product.
75. The composition or combination of any of the preceding numbered paragraphs, further comprising a carrier.
76. The composition or combination of any of the preceding numbered paragraphs, in a shelf-stable formulation.
77. The composition or combination of any of the preceding numbered paragraphs, formulated as a powder, a solution, a pill, a capsule, a concentrate, a tablet, a soft-gel, a thin-film, a liquid, or a syrup.
78. The composition or combination of any of the preceding numbered paragraphs, formulated as a powder.
79. The composition or combination of any of the preceding numbered paragraphs, formulated as a composition comprising at least 10% protein and/or fats by weight.
80. The composition or combination of numbered paragraph 79, wherein the composition comprises at least 20% protein and/or fats by weight.
81. The composition or combination of any of the preceding numbered paragraphs, wherein the composition is chewable or a tablet.
82. The composition or combination of any of the preceding numbered paragraphs, wherein the composition is chewable.
83. The composition or combination of any of the preceding numbered paragraphs, wherein the composition does not comprise a coating or capsule.
84. The composition or combination of any of the preceding numbered paragraphs, formulated with an enteric coating.
85. The composition or combination of any of the preceding numbered paragraphs, provided as a single composition.
86. The composition or combination of any of the preceding numbered paragraphs, provided as a combination of multiple compositions that can be mixed prior to administration or consumption, and/or administered or consumed separately.
87. The composition or combination of any of the preceding numbered paragraphs, not comprising a live microbe.
88. The composition or combination of any of the preceding numbered paragraphs, not comprising a live yeast.
89. The composition or combination of any of the preceding numbered paragraphs, not comprising flour.
90. The composition or combination of any of the preceding numbered paragraphs, further comprising 5-D-fructose dehydrogenase.
91. The composition or combination of any of the preceding numbered paragraphs, not comprising an invertase.
92. The composition or combination of any of the preceding numbered paragraphs, not comprising a beta-fructofuranosidase
93. The composition or combination of any of the preceding numbered paragraphs, not comprising a beta-fructosidase.
94. The composition or combination of any of the preceding numbered paragraphs, not comprising alpha-galactosidase, beta-glucanase, and/or pectinase.
95. The composition or combination of any one of the preceding numbered paragraphs, wherein the composition or combination further comprises a live microbe, e.g., a live probiotic microbe.
96. The composition or combination of numbered paragraph 95, wherein one or more of proteins of the composition or combination is expressed by the live microbe.
97. A nucleic acid comprising a sequence encoding one or more of:
   a. At least one fructan-degrading enzyme;
   b. At least one galactosidase;
   c. At least one sorbitol-degrading enzyme;
   d. At least one mannitol-degrading enzyme;
   e. At least one glucose isomerase; and
   f. At least one mannose isomerase; and
at least one of:
   g. an operably linked heterologous promoter; and
   h. at least one codon optimized for expression in bacteria or yeast.
98. A cell comprising the nucleic acid of numbered paragraph 97.
99. A method of improving digestion, the method comprising consuming, or administering to a subject, the composition or combination of any of numbered paragraphs 1-96.
100. A method of treating a digestive condition, the method comprising administering to a subject, the composition or combination of any of numbered paragraphs 1-96.
101. The method of any of numbered paragraphs 99-100, where administering the composition to a subject comprises directing the subject to consume the composition or combination.
102. The method of any of numbered paragraphs 99-101, wherein administering or consuming comprises chewing or masticating the composition or combination.
103. The method of any of numbered paragraphs 99-102, wherein consumption or administration occurs within 30 minutes of consumption of a meal, or consumption of other food or drink.
104. The method of any of numbered paragraphs 99-102, wherein consumption or administration occurs within 30 minutes prior to consumption of a meal, or consumption of other food or drink.
105. The method of any of numbered paragraphs 99-102, wherein consumption or administration occurs within 20 minutes prior to consumption of a meal, or consumption of other food or drink.
106. The method of any of numbered paragraphs 99-102, wherein consumption or administration occurs within 10 minutes prior to consumption of a meal, or consumption of other food or drink.
107. The method of any of numbered paragraphs 99-102, wherein consumption or administration occurs at least partially during consumption of a meal, or consumption of other food or drink.
108. The method of any of numbered paragraphs 99-102, wherein consumption or administration comprises consuming or ingesting the i) composition or combination and ii) a meal, food, or drink in the same bite or swallow.
109. The method of numbered paragraph 108, wherein the composition or combination has been mixed, sprinkled, dissolved, combined with, or added to at least a portion of the meal, food, or drink.
110. The method of numbered paragraph 108, wherein the composition or combination has been mixed, sprinkled, dissolved, combined with, or added to the meal, food, or drink.
111. The method of any one of numbered paragraphs 108-110, wherein the composition or combination has been mixed, sprinkled, dissolved, combined with, or added to at least a portion of the meal, food, or drink immediately before consumption or administration.
112. The method of any one of numbered paragraphs 108-110, wherein the composition or combination has been mixed, sprinkled, dissolved, combined with, or added to at least a portion of the meal, food, or drink no more than 1 minute before consumption or administration.
113. The method of any one of numbered paragraphs 108-110, wherein the composition or combination has been mixed, sprinkled, dissolved, combined with, or added to at least a portion of the meal, food, or drink no more than 5 minutes before consumption or administration.
114. The method of any one of numbered paragraphs 99-113, wherein the combination or composition comprises a live microbe according to any one of numbered paragraphs 95-96 and whereby the live microbe colonizes the mouth and/or small intestine of the subject.
115. The method of any of numbered paragraphs 99-114, wherein the digestive condition is Irritable Bowel Syndrome (IBS); Inflammatory Bowel Disease (IBD); Non-celiac gluten sensitivity; small intestinal bacterial overgrowth (SIBO); small intestinal fungal overgrowth (SIFO); and/or allium intolerance.
116. The method of any of numbered paragraphs 99-115, wherein the subject is sensitive to one or more FODMAPs.
117. The method of any of numbered paragraphs 99-116, whereby bloating, gas, abdominal pain, abdominal discomfort, nausea, diarrhea, and/or constipation is decreased.

The technology described herein is further illustrated by the following examples which in no way should be construed as being further limiting.

### EXAMPLES

### Example 1

Experiments performed on a healthy individual consistently show remarkable reduction in symptoms when enzyme is used compared to control. The dose of high-fructan foods provided to the subject is enough to induce FODMAP-sensitivity symptoms in nearly all humans. Over the entire length of the testing period, subject has been on a low-FODMAP diet except when ingesting large amounts of fructan administered for testing purposes, in order to avoid interference of gastrointestinal symptoms from other meals. As the test subject is considered healthy, with no known gastrointestinal conditions or complaints, the amount of fructan administered at once was made to be very high so as to induce noticeable symptoms in the subject and aggravate gastrointestinal discomfort at a level that may be comparable to that experienced by individuals sensitive to smaller quantities of ingested fructans. The high-fructan food of choice in this case is shallots. The Monash University application database lists shallots as high in fructan at 6 g. This means that an individual sensitive to fructan will likely experience gastrointestinal symptoms at this serving size and should avoid intake. In the experiments related to the present invention, the healthy subject was administered 130 g of shallots at each testing instance, more than 20 times a serving size considered to cause gastrointestinal distress in individuals sensitive to fructan.

Shallots were fried in oil for seven minutes on medium heat, then administered with or without enzyme mixed into the dish. The enzymes included in the experiment are as follows: (1) exo-inulinase (at activity level of 20,000 units/g) procured from Creative Enzymes; (2) glucose isomerase (5,000 units/g) procured from Creative Enzymes; and (3) endo-inulinase (10,000 units/g) procured from Shaanxi Greenbo Biochem Co., Ltd (Xian, China). Healthy subject then proceeded to track symptoms for the next twelve hours. Each instance of flatulence was noted and timestamped, accompanied by a judgment on magnitude (either “micro,” “small,” “medium,” or “large”). Symptoms like abdominal pain, abdominal discomfort, and bloating were noted when they arose. See Table 11 below for symptoms by category during each experiment. Instances of flatulence within two hours of ingesting the shallots were omitted due to the low likelihood of the fructans having reached the intestines where they could be fermented in so short of a time. Each row indicates a distinct experiment with the 130 g of shallots.

Comparison between the two trials during which no enzyme was given and the four trials accompanied with some combination of enzyme show clear reduction in flatulence (on account of total instances as well as the number of “large” instances) and other symptoms.

TABLE 11

| | | | | | Total | |
|---|---|---|---|---|---|---|
| Enzymes | | | | | Instances | Notes of Other |
| Given | “Micro” | “Small” | “Medium” | “Large” | (Unweighted) | Symptoms |
| None | 1 | 10 | 17 | 2 | 30 | Abdominal discomfort |
| None | 0 | 3 | 9 | 5 | 17 | Abdominal discomfort, |
| | | | | | | bloating, mild pain, |
| | | | | | | visible abdominal |
| | | | | | | distension, flatulence |
| | | | | | | odor (subject reported |
| | | | | | | bloating, distension, |
| | | | | | | and mild pain to have |
| | | | | | | continued the next |
| | | | | | | morning) |
| 1 g exo- | 0 | 4 | 6 | 0 | 10 | None reported |
| inulinase | | | | | | |
| 0.8 g exo- | 0 | 1 | 7 | 0 | 8 | None reported |
| inulinase | | | | | | |
| 1.2 g exo- | 3 | 1 | 1 | 0 | 5 | None reported |
| inulinase; | | | | | | |
| 1 g endo- | | | | | | |
| inulinase; | | | | | | |
| 0.75 g | | | | | | |
| glucose | | | | | | |
| isomerase | | | | | | |
| 1.75 g exo- | 0 | 1 | 5 | 1 | 7 | None reported |
| inulinase; | | | | | | |
| 0.5 g endo- | | | | | | |
| inulinase | | | | | | |

### Example 2

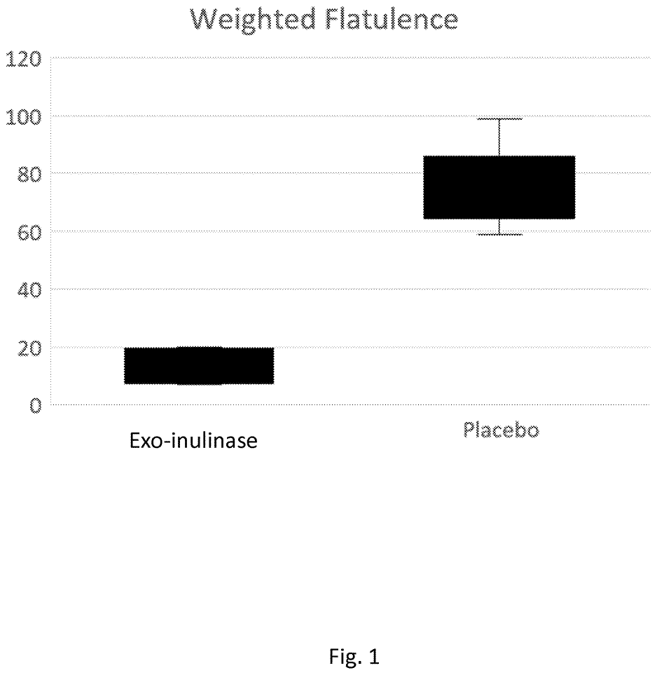

Following the protocol related in Example 1, subjects were administered 260 g servings of steamed shallots with exo-inulinase or a dextrose/maltodextrin placebo. Flatulence symptoms were recorded over a 24 hour period. Administration of exo-inulinase clearly reduced flatulence (on account of total instances as well as the number of “large” instances) (Table 16). The affect on flatulence is shown in FIG. 1, which depicts weighted flatulence scores.

TABLE 16
Flatulence occurrences. Each line is an individual
treatment and 24 hour symptom observation period.

| Treatment | Weighted | Small | Medium | Large |
|---|---|---|---|---|
| Exo-inulinase | 20 | 6 | 7 | 0 |
| Placebo | 99 | 8 | 29 | 11 |
| Placebo | 59 | 8 | 21 | 3 |
| Exo-inulinase | 19 | 4 | 6 | 1 |
| Placebo | 70 | 4 | 27 | 4 |
| Placebo | 71 | 5 | 24 | 6 |
| Exo-inulinase | 9 | 5 | 2 | 0 |
| Exo-inulinase | 7 | 1 | 3 | 0 |
| Exo-inulinase | 8 | 2 | 3 | 0 |
| Placebo | 73 | 11 | 25 | 4 |

### Example 3

Following the protocol related in Example 1, subjects were administered chicory root fiber bars comprising 40 g of fructan/inulin at each testing instance, approximately 200 times the serving size considered to cause gastrointestinal distress in individuals sensitive to fructan. This dose of fructan/inulin will induce FODMAP-sensitivity symptoms in nearly all subjects, regardless of whether they report FODMAP-sensitivity in response to their normal diets.

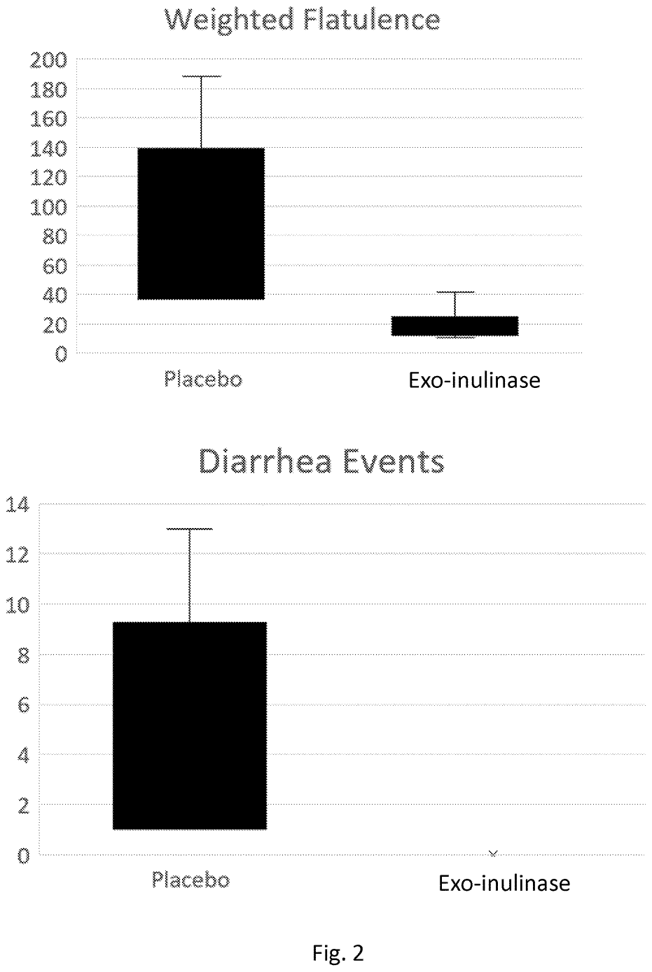

The subjects were administered exo-inulinase exo-inulinase or a dextrose/maltodextrin placebo with the fructan/inulin. Flatulence and diarrhea symptoms were recorded over a 24 hour period (Table 17). Administration of exo-inulinase clearly reduced diarrhea and flatulence (on account of total instances as well as the number of “large” instances). The affect on diarrhea and flatulence is shown in FIG. 2, which depicts diarrhea episodes and weighted flatulence scores.

TABLE 17

| | Weighted | Flatulence | | | Diarrhea |
|---|---|---|---|---|---|
| Treatment | Flatulence | Small | Medium | Large | Events |
| Placebo | 78 | 7 | 22 | 9 | 1 |
| Placebo | 85 | 6 | 26 | 9 | 8 |
| Placebo | 37 | 3 | 14 | 2 | 7 |
| Placebo | 37 | 1 | 6 | 8 | 13 |
| Placebo | 43 | 4 | 6 | 9 | 8 |
| Placebo | 188 | 6 | 31 | 40 | 1 |
| Placebo | 139 | 8 | 28 | 25 | 0 |
| Exo-inulinase | 25 | 8 | 4 | 3 | 0 |
| Exo-inulinase | 18 | 9 | 3 | 1 | 0 |
| Exo-inulinase | 12 | 1 | 4 | 1 | 0 |
| Exo-inulinase | 15 | 2 | 5 | 1 | 0 |
| Exo-inulinase | 11 | 3 | 4 | 0 | 0 |
| Exo-inulinase | 20 | 7 | 4 | 0 | Not |
| | | | | | recorded |
| Exo-inulinase | 42 | 5 | 8 | 7 | Not |
| | | | | | recorded |

What is claimed herein is:

1. A method of improving digestion, the method comprising consuming, or administering to a subject, a composition comprising at least one exo-inulinase.

2. The method of claim 1, wherein the at least one exo-inulinase is an exo-inulinase of, or is obtained from Aspergillus oryzae.

3. The method of claim 1, wherein the exo-inulinase has a sequence with at least 80% sequence identity to one of SEQ ID NOs: 3, 5, 43, or 44.

4. The method of claim 1, wherein the composition further comprises at least one carrier and is in a shelf-stable formulation.

5. The method of claim 1, wherein the composition is formulated as a powder.

6. The method of claim 1, wherein the composition is provided in a kit further comprising a dispenser or dosage guide, the dispenser or dosage guide providing a dose comprising between 500 and 1,000,000 units of enzymatic activity for the at least one exo-inulinase per dose.

7. The method of claim 1, wherein the composition is formulated as a capsule, pill, or tablet, the capsule, pill, or tablet comprising a dose of between 500 and 1,000,000 units of enzymatic activity for each enzyme per dose.

8. The method of claim 1, wherein the composition further comprises at least one of:
at least one endo-inulinase;
at least one galactosidase;
at least one sorbitol-degrading enzyme;
at least one mannitol-degrading enzyme;
at least one glucose isomerase; and
at least one mannose isomerase.

9. The method of claim 8, wherein the at least one endo-inulinase has a sequence with at least 80% sequence identity to one of SEQ ID NOs: 1-2.

10. The method of claim 8, wherein the at least one galactosidase comprises an alpha-galactosidase or at least one lactase.

11. The method of claim 10, wherein the alpha-galactosidase is an alpha-galactosidase of, or is obtained from an Aspergillus spp.

12. The method of claim 10, wherein the alpha-galactosidase is an alpha-galactosidase of, or is obtained from Aspergillus niger.

13. The method of claim 10, wherein the alpha-galactosidase comprises a sequence with at least 80% sequence identity to one of SEQ ID NOs: 11, 12, or 14.

14. The method of claim 1, wherein the composition does not comprise an amyloglucosidase.

15. The method of claim 1, wherein the composition is formulated to comprise at least 10% protein and/or fats by weight.

16. The method of claim 1, wherein the composition does not comprise a coating or capsule.

17. The method of claim 1, wherein consumption or administration occurs within 30 minutes of consumption of a meal, or consumption of other food or drink.

18. The method of claim 1, wherein:
a) consumption or administration occurs at least partially during consumption of a meal, or consumption of other food or drink;
b) consumption or administration comprises consuming or ingesting the i) composition and ii) a meal, food, or drink in the same bite or swallow; or
c) the composition has been mixed, sprinkled, dissolved, combined with, or added to at least a portion of the meal, food, or drink.

19. The method of claim 1, wherein the digestive condition is Irritable Bowel Syndrome (IBS); Inflammatory Bowel Disease (IBD); Non-celiac gluten sensitivity; small intestinal bacterial overgrowth (SIBO); small intestinal fungal overgrowth (SIFO); or allium intolerance.

20. The method of claim 1, wherein the subject is sensitive to one or more FODMAPs.

21. The method of claim 20, wherein the one or more FODMAPs comprise at least one of fructan, sorbitol, and mannitol.

22. The method of claim 1, whereby bloating, gas, abdominal pain, abdominal discomfort, nausea, diarrhea, or constipation is decreased.

23. The method of claim 1, wherein consumption of the composition by a subject decreases bloating, gas, abdominal pain, abdominal discomfort, nausea, diarrhea, or constipation caused by one or more FODMAPs.
